(19) United States
(12) Patent Application Publication
(10) Pub. No.: US 2022/0381786 A1
(43) Pub. Date: Dec. 1, 2022

(54) USE OF DKK-1 INHIBITORS FOR TREATING CANCER

(71) Applicant: Leap Therapeutics, Inc., Cambridge, MA (US)

(72) Inventors: Michael H. Kagey, Arlington, MA (US); Girish Somaia Naik, New York, NY (US); Cynthia A. Sirard, Cambridge, MA (US)

(21) Appl. No.: 17/761,946

(22) PCT Filed: Sep. 18, 2020
(86) PCT No.: PCT/US20/51550

§ 371 Date: Mar. 18, 2022

Related U.S. Application Data
(60) Provisional application No. 62/902,857, filed on Sep. 19, 2019.

(51) Int. Cl. G01N 33/574

(52) U.S. Cl. CPC G01N 33/57484; G01N 33/57446; G01N 33/57442

(57) ABSTRACT

A method of treating a cancer in a subject, in need thereof, comprising determining a DKK1 expression H-score or % positive in a sample of the subject's cancer; and administering a DKK1 antagonist to the subject determined to have the DKK1 expression H-score or % positive equal to or greater than a predetermined value.

### RELATED APPLICATIONS

This application claims the benefit of the filing date of U.S. Provisional Application 62/902,857 filed Sep. 19, 2019. The entire teachings of the above application are incorporated herein by reference.

### BACKGROUND OF THE INVENTION

Cancer is a cellular disorder characterized by uncontrolled or disregulated cellular proliferation, decreased cellular differentiation, inappropriate ability to invade surrounding tissue, and/or ability to establish new growth at ectopic sites. Depending on the specific cancer involved, the treatment for cancer may involve surgery, radiotherapy, chemotherapy or a combination of these treatments. It is estimated that in 2018 in the U.S. Pat. No. 1,735,350 new cases of cancer will be diagnosed and 604,640 people will die from cancer. As such, despite significant advancements in the treatment of cancer, there is a continuing need for new and improved treatments for patients with cancer.

### SUMMARY OF THE INVENTION

In an example embodiment, the present invention is a method of treating a cancer in a subject in need thereof, comprising determining a DKK1 expression by H-score or percent positive in a sample of the subject's cancer; and administering a first amount of a DKK1 inhibitor to the subject determined to have the DKK1 expression H-score or percent positive above a predetermined value.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram representing patient subgroups that, in combination, comprise 134 patients used in the Pooled Analysis of DKK1 H-score by RNAscope, as described in Example 1.

FIG. 2 is superimposition of three plots, each plot representing a tertile of the 134 patients by their DKK1 H-score, of Progression Free Survival (PFS, measured as probability) as a function of the number of days.

FIG. 3 is a table and a plot representing Hazard Ratio (HR) of the pool of the 134 patients, separated by their DKK1 H-score tertiles as well as subgroups of patients defined by the types of cancer and the therapeutic agent(s).

FIG. 4 is a plot of the Standardized Log-Rank Statistics (described herein) as a function of the DKK1 H-score.

FIG. 5 is a superposition of two plots, each plot representing the PFS (expressed as probability) of a subgroup of patients as a function of time.

FIG. 6 is a table and a plot representing Hazard Ratio of the pool of the 134 patients, separated by their optimal DKK1 H-score as well as subgroups of patients defined by the types of cancer and the therapeutic agent(s).

FIG. 7 is a graphical representation of a comparison of different cutpoints that demonstrate that the optimal cutpoint has the best adjusted Hazard Ratio (HR).

FIG. 8 shows two superimposed plots, each representing PFS (expressed as probability) as a function of time of a subgroup of EEC/EOC patients.

FIG. 9 is a table and a plot representing HR for the sub-subgroups of EEC/EOC patients.

FIG. 10 shows two superimposed plots, each representing PFS (expressed as probability) as a function of time of GEJ/GC/EC patients.

FIG. 11 is a table and a plot representing HR for the subgroups of GEJ/GC/EC patients.

FIG. 12 is a bar plot representing maximum percent decrease in size of lesions in 25 evaluable GEJ/GC IO-naïve patients receiving DKN-01/anti-PD-1 therapy.

FIG. 13 shows two superimposed plots, each representing PFS (expressed as probability) as a function of time of a subgroup of IO-naïve GEJ/GC patients receiving DKN-01/anti-PD-1 therapy.

FIG. 14A shows two superimposed plots, each representing PFS (expressed as probability) as a function of time of GEJ/GC/EC patients, calculated using % positive metrics, using the lower boundary of the upper tertile as a cutoff value.

FIG. 14B shows two superimposed plots, each representing PFS (expressed as probability) as a function of time of GEJ/GC/EC patients, calculated using % positive metrics, using the “optimal” cutoff value.

FIG. 15 shows two superimposed plots, each representing PFS (expressed as probability) as a function of time of a subgroup of IO-naïve GEJ/GC patients receiving DKN-01/anti-PD-1 therapy.

FIG. 16A and FIG. 16B are scatter plots showing either H-score (A) or % positive values (B) of Gastric/GEJ IO-refractory patients treated as described herein.

### DETAILED DESCRIPTION OF THE INVENTION

A description of example embodiments of the invention follows.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

Esophagogastric cancer as used herein refers to esophageal cancer and gastric (stomach) cancer (GC).

“Esophageal cancer” (EC) as used herein refers to cancer of the esophagus as well as the gastro-esophageal junction (GEJ). As commonly used in the art, esophageal cancer comprises esophageal squamous cell carcinoma (ESCC) and esophageal adenocarcinoma (EAC). Generally, ESCC refers to cancer that originates in squamous cells, which cells line the esophagus in approximately upper ⅔ of the organ. EAC refers to cancer that originates in gland cells, which replace an area of squamous cells (e.g., in Barrett's esophagus), typically in the lower ⅓ of the esophagus. As such, esophageal adenocarcinoma as used herein refers to adenocarcinoma of the esophagus as well as the gastro-esophageal junction.

In some embodiments, the esophageal adenocarcinoma is recurrent, metastatic, or both. In one embodiment, a recurrent esophageal adenocarcinoma can be recurrent at the primary site of tumor growth (e.g., recurrent tumor growth occurs at the same site). In another embodiment, a recurrent esophageal adenocarcinoma can be recurrent at a site different from the primary site of tumor growth (e.g., the tumor has metastasized). In further embodiments, a recurrent esophageal adenocarcinoma can be recurrent at both the primary site and a site different from the primary site of tumor growth.

As used herein, the terms “recurrent” and “relapsed” are used interchangeably.

As used herein, “gastric cancer” refers to cancer of the stomach. A specific type of gastric cancer is “gastric adenocarcinoma.” An adenocarcinoma is a type of cancerous tumor that is defined as neoplasia of epithelial tissue that has glandular origin, glandular characteristics, or both.

Gastric cancer is the fourth most common cause of cancer-related death in the world, and it remains difficult to cure in Western countries, primarily because most patients present with advanced disease. The stomach begins at the gastroesophageal junction and ends at the duodenum. Histologically, the 90-95% of gastric malignancies are adenocarcinoma. Curative therapy involves surgical resection, most commonly a total or subtotal gastrectomy, with an accompanying lymphadenectomy. The overall 5-year survival rate of patients with resectable gastric cancer ranges from 10% to 30%.

As used herein, “gynecological cancer” refers to cancer of the endometrium (endometrial cancer) and cancer of the ovaries (ovarian cancer). The uterus is lined with a specific tissue called the endometrium. When cancer grows in this lining it is called endometrial cancer. Most cancers of the uterus are endometrial cancers. In certain embodiments, the endometrial cancer is epithelial endometrial cancer (EEC). In another embodiment, the ovarian cancer is epithelial ovarian cancer (EOC).

As used herein, “biliary tract cancer” refer to cancer of the biliary tract. The biliary tract cancer occurs in the bile ducts (tubes that transport bile from the liver) referred to as “cholangiocarcinoma” or gall bladder cancer. Cholangiocarcinoma is classified into different types based on where the cancer occurs in the bile ducts: intrahepatic cholangiocarcinoma occurs in the parts of the bile ducts within the liver and is sometimes classified as a type of liver cancer; hilar cholangiocarcinoma occurs in the bile ducts just outside of the liver. This type is also called perihilar cholangiocarcinoma; and distal cholangiocarcinoma occurs in the portion of the bile duct nearest the small intestine. Gall bladder cancer occur in the gall bladder.

In an example embodiment, the subject is intolerant to at least one (e.g. one, two, three, four, five, etc.) prior treatment regimen. For example, the subject experienced an adverse reaction to the prior treatment regimen and treatment ceased. In another embodiment, the subject is refractory to at least one prior treatment regimen (e.g., the subject no longer responded to a treatment regimen). In other embodiments, the subject is non-responsive to at least one prior treatment regimen. In various embodiments, the subject experienced a combination of the failures described herein to at least one prior treatment regimen, as appropriate. In another embodiment, the subject is “immunooncology-naïve” (IO naïve).

In further embodiments, esophagogastric, gynecological, and biliary tract tumors that express DKK1, as determined by one or more of the various standard mRNA or protein detection methods known in the art, e.g., in situ hybridzidation or immunohistochemistry. Examples of such tumors include the esophageal adenocarcinoma or the gastric adenocarcinoma.

As used herein, a “cutpoint” or “cutpoint value” refers to a measure of the DKK1 expression (for example, in tumor cells, whether H-score value or a “% positive” value, as discussed below), such that when the patients in the subgroup having a measure of expression (H-score or % positive) above the “cutpoint” are administered a DKK1 inhibitor (e.g. DKN-01), these patients show a statistically significant improvement of the progression-free survival (PFS) as compared to the subgroup of patients having their measure of expression below the “cutpoint” value.

As used herein, “an optimal DKK1 expression”, whether expressed by a H-score value or a % positive value (“an optimal H-score”, an “optimal % positive,” or simply “an optimal cutpoint”) refer to a measure of expression of the DKK1 (H-score value or % positive value) at which the absolute value of the “Standardized Log-Rank Statistic” reaches its maximum, where the “Standardized Log-Rank Statistic” is calculated as outlined below.

Log rank statistic for a fixed cutpoint in the range of X1, . . . , Xn is defined by T_{μ} s:

$${T}_{μ} = {T}_{μ} ( a , X ) = {\sum }_{i = 1}^{N} χ \{ {X}_{i} ⩽ μ \} {a}_{i} = {\sum }_{\{ {X}_{i} ⩽ μ \}} {a}_{i} ,$$

where, the log-rank score (a) for observation i is given by:

$${a}_{i} = {a}_{i} ( Z , δ ) = {δ}_{i} - {\sum }_{j = 1}^{η ( Z )} \frac{{δ}_{j}}{( N - {γ}_{j} ( Z ) + 1 )} ,$$

where

$${γ}_{j} ( Z ) = {\sum }_{i = 1}^{N} χ \{ {Z}_{i} ⩽ {Z}_{j} \}$$

is the number of observations died or censored before or at time Zj. Z=(Z₁, . . . , Z_{N}) and δ=(δ₁, . . . , δ_{N}) is the vector for time to event and censoring indicators, respectively. Standardizing the long-rank statistic T_{μ} leads to S_{μ} defined as:

$${S}_{μ} = {S}_{μ} ( a , X ) = \frac{{T}_{μ} - E ( {T}_{μ} \vert X )}{\sqrt{Var ( {T}_{μ} \vert X )}} ,$$

where expectation (E) of T_{μ} under null hypothesis is given by:

$$E ( {T}_{μ} \vert X ) = \frac{{m}_{μ}}{N} {\sum }_{i = 1}^{N} {a}_{i} ,$$

and variance is given by:

$$Var ( {T}_{μ} \vert X ) = \frac{{m}_{μ} {n}_{μ}}{{N}^{2} ( N - 1 )} \{ N {\sum }_{i = 1}^{N} {a}_{i}^{2} - {( {\sum }_{i = 1}^{N} {a}_{i} )}^{2} \}$$

and F_{NX}(μ)=^{N-1}Σᵢ₌₁^{N}χ{Xᵢ≤μ}, denotes the empirical distribution function of X. We restrict the possible cutpoints to an interval μϵ[μ₁,μ₂] according to pre-defined sample quantiles (ε₁,ε₂) of X. μ₁=F_{NX}⁻¹(ε₁) and μ₂=F_{NX}⁻¹(ε₂) where 0<ε₁<ε₂<<1 and F_{NX}⁻¹(t)=min{x|F_{NX}(x)≥t}. The sample sizes in both groups determined by μ are m_{μ}NF_{NX}(μ) and n_{μ}=N−m_{μ}.

The maximum of the absolute value of the standardized statistic (log-rank) S₁ defines a cutpoint estimator, which maximizes the separation of the observations. Using the definitions above, the maximally selected log-rank statistic is defined as:

$$M ( a , X , {μ}_{1 ,} {μ}_{2} ) = {max}_{μ ∈ [ {μ}_{1 ,} {μ}_{2} ]} ( \vert {S}_{μ} ( a , X ) \vert )$$

For full details refer to Torsten Hothorn and Berthold Lausen. “On the exact distribution of maximally selected rank statistics. Computational Statistics & Data Analysis, 43(2): 121-137, June 2003. The teachings of this reference are incorporated herein by reference in their entirety.

The full algorithm implementation is described in maxstat (R) and as such the package “maxstat” was used for this analysis, see Torsten Hothorn (2017). maxstat: Maximally Selected Rank Statistics. R package version 0.7-25, found at the URL https://CRAN.R-project.org/package=maxstat.

In certain embodiments, cancer patients treated with DKN-01-based therapies were grouped as DKK1 H-score “high” if the value of H-score was above the optimal cutpoint and patients who had H-score below the optimal cutpoint were grouped as DKK1 “low”. The optimal cutpoint maximizes the separation of the observations between these two groups.

As used herein, the phrase “above a predetermined value” means “equal to or greater than a predetermined value” when referring to percentiles of values (e.g., “the upper tertile”), and “greater than a predetermined value” when referring to a selected numerical values, such as the “optimal DKK1 expression H-score.”

H-Score and % Positive Values

The level of expression of a gene product of interest, e.g., the expression of DKK1, can be evaluated by methods of immunohistochemistry or in situ hybridization techniques. Convenient semiquantitative measures of the level of expression are computing % positive value (% of cells stained by DKK-1 RNA detecting reagent) or assigning an H-score (or “histo” score) to tumor samples. For H-score, a staining intensity (0, 1+, 2+, or 3+) is determined for each cell in a fixed field. The H-score may then be based on a predominant staining intensity, or more complexly, can include the sum of individual percentages for each intensity level seen. By one method, the percentage of cells at each staining intensity level is calculated, and finally, an H-score is assigned using the following formula:

H-score=[1×(% cells 1+)+2×(% cells 2+)+3×(% cells 3+)]

The final score, ranging from 0 to 300, gives more relative weight to higher-intensity or amount of staining in a given tumor sample. The sample can then be considered positive or negative on the basis of a specific discriminatory threshold. See, for example, Hirsch F R, Varella-Garcia M, Bunn P A Jr, et al: Epidermal growth factor receptor in non-small-cell lung carcinomas: Correlation between gene copy number and protein expression and impact on prognosis. J Clin Oncol 21:3798-3807, 2003; and John T, Liu G, Tsao M-S: Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009. The relevant teachings of these references are herein incorporated by reference.

In various embodiments, an H-score (e.g., a predetermined value of the H-score) can be from 1 to 300, for example, 20 to 100, or 20 to 50. Example predetermined values of H-score are: 20, 25, 30, 35, 40, 45, 50, 55, 60, 65, 70, 75, 80, 85, 90, 95, and 100. In alternative example embodiments, predetermined values of H-score are: 10 or greater, 20 or greater, 30 or greater, 40 or greater, 50 or greater, 60 or greater, 70 of greater, 80 or greater, or 90 or greater. In certain embodiments, the predetermined value of H-score is: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 103, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300.

In alternative embodiments, the measure of DKK1 expression can be a value of the fraction of tumor cells that stain positive for DKK1 (% positive). First a staining amount (0, 1+, 2+, or 3+), based on number of dots in the cell, is determined for each tumor cell in a fixed field. After all neoplastic cells were assigned as “positive,” (e.g. detecting a single staining dot for RNAscope in situ hybridization), the percentage of positive tumor cells is determined by adding up the total neoplastic cells with staining and dividing by the total number of neoplastic cells. % Positive can range from 0 to 100.

In various embodiments, % Positive value (e.g., a predetermined value of % positive) can be from 15% to 50%, for example from 20% to 40%, or 20% to 25%. Example predetermined values of % Positive are: 15% or greater, 20% or greater, 25% or greater, 30% or greater, 35% or greater, 40% or greater, 45% or greater, or 50% of greater. In certain embodiments, the predetermined value of % positive is: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

RNAscope Analysis

One of the methods of computing an H-score of a sample is an RNAscope® in situ hybridization technique developed by and commercially available from Advanced Cell Diagnostics, Inc., as described, for example, at the URL https://acdbio.com/. This technique relies on an optical signal from a hybridization probe cognate to the mRNA of interest. The signal can be detected either by a bright-field or epifluorescent microscopy. The technique permits detection of a single molecule. See, for example, RNAscope: A Novel In Situ RNA Analysis Platform for Formalin-Fixed Paraffin-Embedded Tissues. Wang F, Flanagan J, Su N, Wang L C, Bui S, Nielson A, Wu X, Vo H T, Ma X J, Luo Y (2012). J of Mol Diagnostics, 14(1):22-29.

Alternative Methods of Detecting Biomarkers

In addition to RNAscope® techniques, alternative methods of detecting biomarkers, e.g., the level of a gene product expression, such as the DKK1 expression level, can be used.

Immunohistochemical (IHC) staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, in some embodiments, polyclonal antisera, and in some embodiments, monoclonal antibodies specific for each marker are used to detect expression. As discussed in greater detail below, the antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromagenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al, Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE® and SPECTRUM GREEN® and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, D-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al. Methods for the Preparation of Enzyme-Antibody Conjugates for use in Enzyme Immunoassay, in Methods in Enzym. (ed. J. Langone & H. Van Vunakis), Academic press, New York, 73: 147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor [e.g., orthophenylene diamine (OPD) or 3,3′,5,5′-tetramethyl benzidine hydrochloride (TMB)]. 3,3-Diaminobenzidine (DAB) may also be used to visualize the HRP-labeled antibody;

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate (e.g., 4-methylumbelliferyl\hat{ }-D-galactosidase).

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice verse. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the standard sample preparation procedures known to a person of ordinary skill in the art, further treatment of the tissue section prior to, during or following IHC may be desired. For example, epitope retrieval methods, such as heating the tissue sample in citrate buffer may be carried out [see, e.g., Leong et al. Appl. Immunohistochem. 4(3):201 (1996)].

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation.

The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

IHC may be combined with morphological staining, either prior to or thereafter. After deparaffinization, the sections mounted on slides may be stained with a morphological stain for evaluation. The morphological stain to be used provides for accurate morphological evaluation of a tissue section. The section may be stained with one or more dyes each of which distinctly stains different cellular components. In one embodiment, hematoxylin is use for staining cellular nucleic of the slides. Hematoxylin is widely available. An example of a suitable hematoxylin is Hematoxylin II (Ventana). When lighter blue nuclei are desired, a bluing reagent may be used following hematoxylin staining. One of skill in the art will appreciate that staining may be optimized for a given tissue by increasing or decreasing the length of time the slides remain in the dye.

Automated systems for slide preparation and IHC processing are available commercially. The Ventana® BenchMark XT system is an example of such an automated system. After staining, the tissue section may be analyzed by standard techniques of microscopy. Generally, a pathologist or the like assesses the tissue for the presence of abnormal or normal cells or a specific cell type and provides the loci of the cell types of interest. Thus, for example, a pathologist or the like would review the slides and identify normal cells (such as normal lung cells) and abnormal cells (such as abnormal or neoplastic lung cells). Any means of defining the loci of the cells of interest may be used (e.g., coordinates on an X-Y axis).

Other techniques for biomarker detection are known in the art, including but not limited to nucleic acid detection methods (including but not limited to PCR, sequencing, rtPCT, RNA-seq, microarray analysis, SAGE, Mass ARRAY technique and FISH) and protein detection methods (including but not limited to mass spec, western blotting). Detecting amplification of the c-met gene is achieved using certain techniques known to those skilled in the art. For example, comparative genome hybridization may be used to produce a map of DNA sequence copy number as a function of chromosomal location. See, e.g., Kallioniemi et al. (1992) Science 258:818-821. Amplification of the c-met gene may also be detected, e.g., by Southern hybridization using a probe specific for the c-met gene or by real-time quantitative PCR. In certain embodiments, detecting amplification of the c-met gene is achieved by directly assessing the copy number of the c-met gene, for example, by using a probe that hybridizes to the c-met gene. For example, a FISH assay may be performed. In certain embodiments, detecting amplification of the c-met gene is achieved by indirectly assessing the copy number of the c-met gene, for example, by assessing the copy number of a chromosomal region that lies outside the c-met gene but is co-amplified with the c-met gene. Biomarker expression may also be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

DKK1 Antibody

DKK1 antibodies have been described previously (see, e.g., U.S. Pat. Nos. 8,148,498 and 7,446,181, incorporated by reference herein in their entireties). The DKK1 antibody or antigen-binding fragment thereof disclosed herein relates to human engineered antibodies that bind to a human DKK1 comprising the amino acid sequence set for in SEQ ID NO: 22, or fragments thereof. The present DKK1 antibodies are therapeutically useful DKK1 antagonists possessing a number of desirable properties. For example, the DKK1 antibodies block DKK1 mediated inhibition of alkaline phosphatase, a marker or osteoblast activity, as well as treat various types of cancer (e.g., non-small cell lung cancer).

A full-length antibody as it exists naturally is an immunoglobulin molecule comprising 2 heavy (H) chains and 2 light (L) chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (“FR”). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions is in accordance with the well-known Kabat numbering convention.

Light chains are classified as kappa or lambda, and are characterized by a particular constant region as known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

As used herein, the term “monoclonal antibody” (Mab) refers to an antibody that is derived from a single copy or clone including, for example, any eukaryotic, prokaryotic, or phage clone, and not the method by which it is produced. Mabs of the present invention preferably exist in a homogeneous or substantially homogeneous population. Complete Mabs contain 2 heavy chains and 2 light chains.

Unless specified otherwise, the term “DKK1 antibody” encompasses both a full-length antibody as well as an antigen binding-fragment of the DKK1 antibody.

“Antigen-binding fragments” of such monoclonal antibodies include, for example, Fab fragments, Fab′ fragments, F(ab′)₂ fragments, and single chain Fv fragments as well as bispecific and/or multivalent antibodies that may utilize the DKN-01 CDRs. Monoclonal antibodies and antigen-binding fragments thereof can be produced, for example, by recombinant technologies, phage display technologies, synthetic technologies, e.g., CDR-grafting, or combinations of such technologies, or other technologies known in the art. For example, mice can be immunized with human DKK1 or fragments thereof, the resulting antibodies can be recovered and purified, and determination of whether they possess binding and functional properties similar to or the same as the antibody compounds disclosed herein can be assessed by the methods known in the art. Antigen-binding fragments can also be prepared by conventional methods. Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15, ISBN 0-87969-314-2.

Monoclonal DKK1 antibodies disclosed herein are engineered to comprise framework regions that are substantially human or fully human surrounding CDRs derived from a non-human antibody. “Antigen-binding fragments” of such human engineered antibodies include, for example, Fab fragments, Fab′ fragments, F(ab′)₂ fragments, and single chain Fv fragments. “Framework region” or “framework sequence” refers to any one of framework regions 1 to 4. Human engineered antibodies and antigen-binding fragments thereof encompassed by the antibodies disclosed herein include molecules wherein any one or more of framework regions 1 to 4 is substantially or fully human, i.e., wherein any of the possible combinations of individual substantially or fully human framework regions 1 to 4, is present. For example, this includes molecules in which framework region 1 and framework region 2, framework region 1 and framework region 3, framework region 1, 2, and 3, etc., are substantially or fully human. Substantially human frameworks are those that have at least about 80% sequence identity to a known human germline framework sequence. Preferably, the substantially human frameworks have at least about 85%, about 90%, about 95%, or about 99% sequence identity to a known human germline framework sequence.

Human engineered antibodies in addition to those disclosed herein exhibiting similar functional properties can be generated using several different methods. The specific antibody compounds disclosed herein can be used as templates or parent antibody compounds to prepare additional antibody compounds. In one approach, the parent antibody compound CDRs are grafted into a human framework that has a high sequence identity with the parent antibody compound framework. The sequence identity of the new framework will generally be at least about 80%, at least about 85%, at least about 90%, at least about 95%, or at least about 99% identical to the sequence of the corresponding framework in the parent antibody compound. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful in humanizing mouse antibodies include U.S. Pat. Nos. 4,816,397; 5,225,539, and 5,693,761; computer programs ABMOD and ENCAD as described in Levitt (1983) J. Mol. Biol. 168:595-620; and the method of Winter and co-workers (Jones et al. (1986) Nature 321:522-525; Riechmann et al. (1988) Nature 332:323-327; and Verhoeyen et al. (1988) Science 239:1534-1536). Methods for identifying residues to consider for back-mutation are known in the art (see, e.g., U.S. Pat. No. 8,148,498).

The DKK1 antibody administered in the method of treatment described herein comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, HCDR1 has the amino sequence of SEQ ID NO:4, and HCDR2 has the amino sequence of SEQ ID NO:5.

In one embodiment, the DKK1 antibody comprises a LCDR1 having the amino sequence of SEQ ID NO:1, LCDR2 having the amino sequence of SEQ ID NO:2, LCDR3 having the amino sequence of SEQ ID NO:3, HCDR1 having the amino sequence of SEQ ID NO:4, HCDR2 having the amino sequence of SEQ ID NO:5, and HCDR3 having the amino sequence of SEQ ID NO:6.

In a another embodiment, the DKK1 antibody comprises a LCVR having the amino acid sequence of SEQ ID NO: 7 and a HCVR having the amino acid sequence of SEQ ID NO: 8. In a particular embodiment, the LCVR comprises the amino acid sequence of SEQ ID NO: 11 and the HCVR comprises the amino acid sequence of SEQ ID NO: 12.

In further embodiments, the DKK1 antibody comprises a heavy chain (HC) having the amino acid sequence of SEQ ID NO: 17 and a light chain (LC) having the amino acid sequence of SEQ ID NO: 18. The DKK1 antibody or antigen binding-fragment thereof comprising the HC and LC amino acid sequence of SEQ ID NO: 17 and SEQ ID NO: 18, respectively, is referred to herein as DKN-01. In particular, DKN-01 has the molecular/empirical formula C₆₃₉₄ H₉₈₁₀ N₁₆₉₈ O₂₀₁₂ S₄₂ and a molecular weight of 144015 Daltons (intact).

In certain embodiments, the DKK1 antibody disclosed herein is an IgG4 antibody with a neutralizing activity against human DKK1 comprising the sequence set forth in SEQ ID NO: 22, of a fragment thereof. For example, canonical Wnt signaling is important for osteoblast differentiation and activity. Wnt-3a combined with BMP-4 induces multipotent mouse C2C12 cells to differentiate into osteoblasts with a measurable endpoint of alkaline phosphatase (“AP”), a marker of osteoblast activity. DKK1, an inhibitor of canonical Wnt signaling, inhibits the differentiation and production of AP. Neutralizing DKK1 antibodies prevent DKK1-mediated inhibition of AP. Antibodies which block DKK1 inhibitory activity prevent the loss of AP activity (see U.S. Pat. No. 8,148,498). In a particular embodiment, the DKK1 antibody possessing neutralizing activity is DKN-01, which is an IgG4 antibody.

The DKK1 antibodies disclosed herein possess high affinity (Kd) to DKK1 (e.g., human DKK1, SEQ ID NO: 22), as described in U.S. Pat. No. 8,148,498. For example, the present DKK1 antibodies possess a Kd of between 0.5×10⁻¹² M and 3.0×10⁻¹¹ M, at 37° C.

### Pembrolizumab

Pembrolizumab is a potent humanized IgG4 mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Pembrolizumab has an acceptable preclinical safety profile and is in clinical development as an IV immunotherapy for advanced malignancies. Known by its trade name Keytruda™, pembrolizumab is indicated for the treatment of patients across a number of indications. Furthermore, pembrolizumab is being investigated in patients with gastrointestinal cancers, including esophageal cancer. Although preliminary, promising findings have been seen in patients with esophageal cancer (Bilgin et al. Targeting the PD-1 pathway: a new hope for gastrointestinal cancers. Curr Med Res Opin. 2017; 33(4):749-759; lams et al. Neoadjuvant Treatment for Locally Invasive Esophageal Cancer. World J Surg. 2017 Mar. 7 [Epub ahead of print]; Chau et al. Interim safety and clinical activity in patients (pts) with advanced gastric or gastroesophageal junction (G/GEJ) adenocarcinoma from a multicohort phase I study of ramucirumab (R) plus pembrolizumab (P). J Clin Oncol. 35, 2017 (suppl 4S; abstract 102)). In May 2017 pembrolizumab was approved for any unresectable or metastatic solid tumor with certain genetic qualities without regard to the tissue type or site of the tumor. In September 2017, pembrolizumab was approved for treating gastric cancer in patients with recurrent locally advanced or metastatic, gastric or gastroesophageal junction adenocarcinoma whose tumors express PD-L1.

Tumors that have mutations that cause DNA mismatch repair, which often results in microsatellite instability, tend to generate many mutated proteins that could serve as tumor antigens; pembrolizumab appears to facilitate clearance of any such tumor by the immune system, by preventing the self-checkpoint system from blocking the clearance.

As used herein, the term “taxanes” includes paclitaxel, docetaxel, carbazitaxel, and their derivatives that possess antineoplastic properties. For example, “paclitaxel” includes both naturally derived and chemically synthesized paclitaxel. Paclitaxel is sold as TAXOL®. Derivatized paclitaxels suitable for use in the invention described herein include deoxygenated paclitaxel compounds such as those described in U.S. Pat. No. 5,440,056, albumin-bound paclitaxel (ABRAXANE), DHA-paclitaxel, and PG-paclitaxel. Chemical formulas for paclitaxel and derivatives thereof are known and described in the art. Other taxane compounds are disclosed in “Synthesis and Anticancer Activity of Taxol other Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elvesier, Amsterdam 1986), pp. 219-235. See also, for example, U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506. The term “docetaxel” includes both naturally derived and chemically synthesized compounds docetaxel. Docetaxel is sold as TAXOTERE®.

Modes of Administration

The DKK1 antibody and other therapeutics agents used in combination with the DKK1 antibody (e.g., pembrolizumab, paclitaxel, cisplatin, gemcitabine etc.) for use in the methods or compositions of the invention can be formulated for parenteral, oral, transdermal, sublingual, buccal, rectal, intranasal, intrabronchial or intrapulmonary administration.

For parenteral administration, the compounds for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For oral administration the compounds can be of the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the methods or compositions of the invention can be in the form of tablets or lozenges formulated in a conventional manner.

For rectal administration, the compounds for use in the methods or compositions of the invention can be in the form of suppositories.

For sublingual administration, tablets can be formulated in conventional manner.

For intranasal, intrabronchial or intrapulmonary administration, conventional formulations can be employed.

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use in the method of the invention can be administered in the form of microparticles, for example, by injection or in the form of wafers or discs by implantation. Various methods of formulating controlled release drug preparations are known in the art.

Administration of a compound (e.g., the DKK1 antibody alone or in combination with one or more additional therapeutic agent), or pharmaceutically acceptable salt thereof, or a composition comprising one or more compound (or pharmaceutical salt thereof) of the invention useful to practice the methods described herein, can be continuous, hourly, four times daily, three time daily, twice daily, once daily, once every other day, twice weekly, once weekly, once every two weeks, once a month, or once every two months, or longer, or some other intermittent dosing regimen.

Examples of administration of a compound, or a composition comprising one or more compound (or pharmaceutical salt thereof) of the invention include peripheral administration. Examples of peripheral administration include oral, subcutaneous, intraperitoneal, intramuscular, intravenous, rectal, transdermal, or intranasal forms of administration.

As used herein, peripheral administration includes all forms of administration of a compound or a composition comprising a compound of the instant invention which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

Combination Therapy

The DKK1 antibody and one or more second therapeutic agents (e.g., pembrolizumab, paclitaxel, cisplatin, gemcitabine etc) for use in the methods or compositions of the invention can be formulated separately or in combination for parenteral, oral, transdermal, sublingual, buccal, rectal, intranasal, intrabronchial or intrapulmonary administration.

The DKK1 antibody disclosed herein can be used for treating an esophagogastric cancer (e.g. esophageal cancer or gastric adenocarcinoma) in combination with pembrolizumab. Such combination administration can be by means of a single dosage form which includes a DKK1 antibody and pembrolizumab, such single dosage form including a tablet, capsule, spray, inhalation powder, injectable liquid or the like. Combination administration can comprise a further additional agent (e.g., chemotherapeutic agent) in addition to the single dosage form. Alternatively, combination administration can be by means of administration of two different dosage forms, with one dosage form containing a DKK1 antibody, and the other dosage form including a second amount of pembrolizumab. In this instance, the dosage forms may be the same or different. Without wishing to limit combination therapies, the following exemplifies certain combination therapies which may be employed. It is understood that additional chemotherapeutic agents beyond the required second amount of pembrolizumab can be employed in the method described herein.

The second amount of pembrolizumab can be administered before, simultaneously with, or after the administration of a DKK1 antibody. Accordingly, a DKK1 antibody and pembrolizumab can be administered together in a single formulation or can be administered in separate formulations, e.g., either simultaneously or sequentially, or both. For example, if a DKK1 antibody and pembrolizumab are administered sequentially in separate compositions, the DKK1 antibody can be administered before or after pembrolizumab. The duration of time between the administration of a DKK1 antibody and the second amount of pembrolizumab will be easily determined by a person of ordinary skill in the art. In certain embodiments, the DKK1 antibody can precede or follow pembrolizumab immediately, or after some duration of time deemed to be appropriate by a skilled practitioner.

In addition, the DKK1 antibody and the second amount of pembrolizumab may or may not be administered on similar dosing schedules. For example, the DKK1 antibody and pembrolizumab may have different half-lives and/or act on different time-scales such that the DKK1 antibody is administered with greater frequency than pembrolizumab or vice-versa. For example, the DKK1 antibody and pembrolizumab can be administered together (e.g., in a single dosage or sequentially) on one day, followed by administration of only the chemotherapeutic agent (or a different chemotherapeutic) a set number of days later. The number of days in between administration of therapeutic agents can be appropriately determined according to the safety, pharmacokinetics and pharmacodynamics of each drug. Either the DKK1 antibody or pembrolizumab can be administered acutely or chronically.

In a particular embodiment, the treatment period for the combination treatment of DKN-01 and pembrolizumab is a 21-Day cycle which can be repeated until the patient is determined to not be gaining any clinical benefit from the combination therapy. For example, the patient can undergo from about one cycle to about 30 cycles of treatment (e.g., 1, 2, 3, 4, 5, 6, 7, 8, 9, 10, 11, 12, 13, 14, 15, 16, 7, 18, 19, 20, 21, 22, 23, 24, 25, 26, 27, 28, 29, 30). In another embodiment, the subject is being treated for a gynecological cancer. Treatment comprises a combined administration of a DKK1 antibody, such as DKN-01, and paclitaxel, following the clinical trials described herein.

As used herein, an “effective amount” refers to an amount of a therapeutic agent or a combination of therapeutic agents that is therapeutically or prophylactically sufficient to treat the target disorder. An effective amount will depend on the age, gender, and weight of the patient, the current medical condition of the patient, and the nature of the esophageal or gastric cancer being treated. Those of skill in the art will be able to determine appropriate dosages depending on these and other factors.

In an example embodiment, a subject in need thereof receives a monotherapy (i.e. is being administered a first amount of a first therapeutic agent), so that the first amount of the first therapeutic agent is an effective amount. In another example embodiment, a subject in need thereof receives a combination therapy, e.g. is being administered a first amount of a first therapeutic agent and a second amount of a second therapeutic agent, so that the first amount and the second amount, in combination, is an effective amount. In further embodiment, a combination therapy can employ a third amount of a third therapeutic agent, so that the first amount, the second amount, and the third amount, in combination, is an effective amount.

An effective amount can be achieved in the methods or compositions of the invention by coadministering a first amount of a DKK1 antibody (or a pharmaceutically acceptable salt, hydrate or solvate thereof) and a second amount of pembrolizumab. In one embodiment, the DKK1 antibody and pembrolizumab are each administered in a respective effective amount (e.g., each in an amount which would be therapeutically effective if administered alone). In another embodiment, the DKK1 antibody and pembrolizumab each is administered in an amount that, alone, does not provide a therapeutic effect (a sub-therapeutic dose). In yet another embodiment, the DKK1 antibody can be administered in an effective amount, while pembrolizumab is administered in a sub-therapeutic dose. In still another embodiment, the DKK1 antibody can be administered in a sub-therapeutic dose, while pembrolizumab is administered in an effective amount.

Suitable doses per administration for a DKK1 antibody include doses of about or greater than about 15 mg, about 20 mg, about 30 mg, about 40 mg, about 50 mg, about 100 mg, about 150 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, or about 3,000 mg. Each suitable dose can be administered over a period time deemed appropriate by a skilled practitioner. For example, each suitable dose can be administered over a period of about 30 minutes and up to about 1 hour, about 2 hours, about 3, hours, about 4 hours, about 5 hours, about 6 hours, about 7 hours, or about 8 hours. In a specific embodiment, a suitable does for the DKK1 antibody (e.g., DKN-01) can from about 50 mg to about 300 mg (such as 50 mg, 75 mg, 100 mg, 125 mg, 150 mg, 175 mg, 200 mg, 225 mg, 250 mg, 275 mg or 300 mg). The selected dose can be administered intravenously over a period of about 30 minutes to about 2 hours. In a particular embodiment, a suitable dose for DKK1 antibody can be about 150 mg administered over a period of about 30 minutes and up to about 2 hours. Another suitable dose for the DKK1 antibody can be about 300 mg administered over a period of about 30 minutes and up to about 2 hours. Administration of these doses over the recited period of time can be accomplished using an intravenous route.

Suitable doses per administration for pembrolizumab can be determined based on the recommended dosing found on the label. For example, a suitable dose per administration of pembrolizumab is from about 50 mg to about 200 mg intravenously over at least a 30 minute period. This administration can be repeated every three weeks. In a particular embodiment, a suitable dose per administration is about 200 mg over a 30 minute infusion period using an intravenous route. This dose can be repeated every three weeks. Other suitable doses of pembrolizumab include 2 mg/kg Q3W (every three weeks), 10 mg/kg Q3W (every three weeks), and 10 mg/kg Q2W (every two weeks). In a particular embodiment, the dose of pembrolixumab is 200 mg intravenously. In one aspect, the 200 mg is administered over 30 minutes.

Suitable doses per administration for taxanes (e.g., paclitaxel) can be determined based on the recommended dosing found on the label. For example, a suitable dose per administration of paclitaxel is from about 200 mg/m2 to about 20 mg/m². In a particular embodiment, the dose of paclitaxel is 80 mg/m². The taxane (e.g., paclitaxel) can be administered intravenously. Intravenous administration can be over about one hour.

Suitable doses per administration for gemcitabine can be determined based on the recommended dosing found on the label. For example, a suitable dose per administration of gemcitabine is from about 2000 mg/m² to about 500 mg/m². In a particular embodiment, the dose of gemcitabine is 1000 mg/m².

Suitable doses per administration for cisplatin can be determined based on the recommended dosing found on the label. For example, a suitable dose per administration of cisplatin is from about 10 mg/m2 to about 40 mg/m²In a particular embodiment, the dose of cisplatin is 20 mg/m².

As used herein, the term “subject” refers to a mammal, preferably a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like). The terms “subject” and “patient” are used interchangeably herein. In a particular embodiment, the subject has been previously treated with an anti-PD-1/PD-L1 monoconal antibody (e.g., pembrolizumab, nivolumab atezolisumag, durvalumab or avelumab) and the subject's disease is refractory to such prior treatment.

As used herein “treating” includes achieving, partially or substantially, delaying, inhibiting or preventing the progression of clinical indications related to the esophageal cancer or gastric adenocarcinoma. For example, “treating” includes reduction in tumor growth, or prevention of further growth, as detected by standard imaging methods known in the art, including, for example, computed tomography (CT) scan, magnetic resonance imaging (MRI), chest x-ray, and CT/positron emission tomography (CT/PET) scans, and evaluated according to guidelines and methods known in the art. For example, responses to treatment can be evaluated through the Response Evaluation Criteria in Solid Tumors (RECIST) (Revised RECIST Guideline version 1.1; see Eisenhauer et al., Eur. J. Cancer 45(2):228-47, 2009). Thus, in some embodiments, “treating” refers to a Complete Response (CR), which is defined according to the RECIST guideline as the disappearance of all target lesions, or a Partial Response (PR), which is defined as at least a 30% decrease in the sum of diameter of target lesions, taking as reference the baseline sum diameters. Other means for evaluating tumor response to treatment include evaluation of tumor markers and evaluation of performance status (e.g., assessment of creatinine clearance; see Cockcroft and Gault, Nephron. 16:31-41, 1976).

Pharmaceutical Composition

The DKK1 antibody and pembrolizumab can be incorporated into pharmaceutical compositions suitable for administration. Such compositions typically comprise the antibody, or pembrolizumab, or one or more additional chemotherapeutic agents, in any combination, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a DKK1 antibody alone or in combination with pembrolizumab, DKK1 antibody in combination with paclitaxel, DKK1 antibody in combination with gemcitabine and cisplatin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid-derivatives. Transmucosal administration can be accomplished through the use of nasal sprays or suppositories.

For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### SEQUENCES

The following are sequences of the DKN-01 antibody that can be employed in the practice of the various example embodiments described herein.

LCDR1
(SEQ ID NO: 1)
His Ala Ser Asp Ser Ile Ser Asn Ser Leu His
LCDR2
(SEQ ID NO: 2)
Tyr Xaa Arg Gln Ser Xaa Gln
wherein Xaa at position 2 is Gly or Ala; and Xaa at position 6 is Ile or Glu
LCDR3
(SEQ ID NO: 3)
Gln Gln Ser Xaa Ser Trp Pro Leu His
wherein Xaa at position 4 is Glu or Ala
HCDR1
(SEQ ID NO: 4)
Gly Phe Thr Phe Ser Ser Tyr Thr Met Ser
HCDR2
(SEQ ID NO: 5)
Thr Ile Ser Gly Gly Gly Phe Gly Thr Tyr Tyr Pro Asp Ser Val Lys
HCDR3
(SEQ ID NO: 6)
Pro Gly Tyr Xaa Asn Tyr Tyr Phe Asp Ile
wherein Xaa at position 4 is His or Asn
LCVR
(SEQ ID NO: 7)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Xaa Arg Gln Ser Xaa Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Xaa Ser Trp Pro Leu His Phe Gly Gly Gly Thr Lys Val Glu Ile Lys
wherein Xaa at position 51 is Gly or Ala; Xaa at position 55 is Ile or Glu and Xaa at position
92 is Glu or Ala.
HCVR
(SEQ ID NO: 8)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Aia Val Tyr Tyr Cys Ala Arg Pro Gly Tyr Xaa Asn Tyr Tyr
Phe Asp Ile Trp Gly Gln Gly Thr Thr Val Thr Val Ser Ser
wherein Xaa at position 102 is His or Asn
LCVR
(SEQ ID NO: 9)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Glu Ser Trp Pro Leu His Phe Gly Gly Gly Thr Lys Val Glu Ile Lys
HCVR
(SEQ ID NO: 10)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Pro Gly Tyr His Asn Tyr Tyr
Phe Asp Ile Trp Gly Gln Gly Thr Thr Val Thr Val Ser Ser
LCVR
(SEQ ID NO: 11)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Glu Ser Trp Pro Leu His Phe Gly Gly Gly Thr Lys Val Glu Ile Lys
HCVR
(SEQ ID NO: 12)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Pro Gly Tyr Asn Asn Tyr Tyr
Phe Asp Ile Trp Gly Gln Gly Thr Thr Val Thr Val Ser Ser
LCVR
(SEQ ID NO: 13)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Ala Ser Trp Pro Leu His Phe Gly Gly Gly Thr Lys Val Glu Ile Lys
LCVR
(SEQ ID NO: 14)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Glu Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Ala Ser Trp Pro Leu His Phe Gly Gly Gly Thr Lys Val Glu Ile Lys
HC
(SEQ ID NO: 15)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Pro Gly Tyr His Asn Tyr Tyr
Phe Asp Ile Trp Gly Gln Gly Thr Thr Val Thr Val Ser Ser Ala Ser Thr Lys Gly Pro Ser Val
Phe Pro Leu Ala Pro Cys Ser Arg Ser Thr Ser Glu Ser Thr Ala Ala Leu Gly Cys Leu Val Lys
Asp Tyr Phe Pro Glu Pro Val Thr Val Ser Trp Asn Ser Gly Ala Leu Thr Ser Gly Val His Thr
Phe Pro Ala Val Leu Gln Ser Ser Gly Leu Tyr Ser Leu Ser Ser Val Val Thr Val Pro Ser Ser
Ser Leu Gly Thr Lys Thr Tyr Thr Cys Asn Val Asp His Lys Pro Ser Asn Thr Lys Val Asp
Lys Arg Val Glu Ser Lys Tyr Gly Pro Pro Cys Pro Pro Cys Pro Ala Pro Glu Ala Ala Gly Gly
Pro Ser Val Phe Leu Phe Pro Pro Lys Pro Lys Asp Thr Leu Met Ile Ser Arg Thr Pro Glu Val
Thr Cys Val Val Val Asp Val Ser Gln Glu Asp Pro Glu Val Gln Phe Asn Trp Tyr Val Asp
Gly Val Glu Val His Asn Ala Lys Thr Lys Pro Arg Glu Glu Gln Phe Asn Ser Thr Tyr Arg
Val Val Ser Val Leu Thr Val Leu His Gln Asp Trp Leu Asn Gly Lys Glu Tyr Lys Cys Lys
Val Ser Asn Lys Gly Leu Pro Ser Ser Ile Glu Lys Thr Ile Ser Lys Ala Lys Gly Gln Pro Arg
Glu Pro Gln Val Tyr Thr Leu Pro Pro Ser Gln Glu Glu Met Thr Lys Asn Gln Val Ser Leu Thr
Cys Leu Val Lys Gly Phe Tyr Pro Ser Asp Ile Ala Val Glu Trp Glu Ser Asn Gly Gln Pro Glu
Asn Asn Tyr Lys Thr Thr Pro Pro Val Leu Asp Ser Asp Gly Ser Phe Phe Leu Tyr Ser Arg
Leu Thr Val Asp Lys Ser Arg Trp Gln Glu Gly Asn Val Phe Ser Cys Ser Val Met His Glu
Ala Leu His Asn His Tyr Thr Gln Lys Ser Leu Ser Leu Ser Leu Gly
LC
(SEQ ID NO: 16)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Glu Ser Trp Pro Leu His Phe Gly Gly Gly Thr Lys Val Glu Ile Lys
Arg Thr Val Ala Ala Pro Ser Val Phe Ile Phe Pro Pro Ser Asp Glu Gln Leu Lys Ser Gly Thr
Ala Ser Val Val Cys Leu Leu Asn Asn Phe Tyr Pro Arg Glu Ala Lys Val Gln Trp Lys Val
Asp Asn Ala Leu Gln Ser Gly Asn Ser Gln Glu Ser Val Thr Glu Gln Asp Ser Lys Asp Ser
Thr Tyr Ser Leu Ser Ser Thr Leu Thr Leu Ser Lys Ala Asp Tyr Glu Lys His Lys Val Tyr Ala
Cys Glu Val Thr His Gln Gly Leu Ser Ser Pro Val Thr Lys Ser Phe Asn Arg Gly Glu Cys
HC
(SEQ ID NO: 17)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Pro Gly Tyr Asn Asn Tyr Tyr
Phe Asp Ile Trp Gly Gln Gly Thr Thr Val Thr Val Ser Ser Ala Ser Thr Lys Gly Pro Ser Val
Phe Pro Leu Ala Pro Cys Ser Arg Ser Thr Ser Glu Ser Thr Ala Ala Leu Gly Cys Leu Val Lys
Asp Tyr Phe Pro Glu Pro Val Thr Val Ser Trp Asn Ser Gly Ala Leu Thr Ser Gly Val His Thr
Phe Pro Ala Val Leu Gln Ser Ser Gly Leu Tyr Ser Leu Ser Ser Val Val Thr Val Pro Ser Ser
Ser Leu Gly Thr Lys Thr Tyr Thr Cys Asn Val Asp His Lys Pro Ser Asn Thr Lys Val Asp
Lys Arg Val Glu Ser Lys Tyr Gly Pro Pro Cys Pro Pro Cys Pro Ala Pro Glu Ala Ala Gly Gly
Pro Ser Val Phe Leu Phe Pro Pro Lys Pro Lys Asp Thr Leu Met Ile Ser Arg Thr Pro Glu Val
Thr Cys Val Val Val Asp Val Ser Gln Glu Asp Pro Glu Val Gln Phe Asn Trp Tyr Val Asp
Gly Val Glu Val His Asn Ala Lys Thr Lys Pro Arg Glu Glu Gln Phe Asn Ser Thr Tyr Arg
Val Val Ser Val Leu Thr Val Leu His Gln Asp Trp Leu Asn Gly Lys Glu Tyr Lys Cys Lys
Val Ser Asn Lys Gly Leu Pro Ser Ser Ile Glu Lys Thr Ile Ser Lys Ala Lys Gly Gln Pro Arg
Glu Pro Gln Val Tyr Thr Leu Pro Pro Ser Gln Glu Glu Met Thr Lys Asn Gln Val Ser Leu Thr
Cys Leu Val Lys Gly Phe Tyr Pro Ser Asp Ile Ala Val Glu Trp Glu Ser Asn Gly Gln Pro Glu
Asn Asn Tyr Lys Thr Thr Pro Pro Val Leu Asp Ser Asp Gly Ser Phe Phe Leu Tyr Ser Arg
Leu Thr Val Asp Lys Ser Arg Trp Gln Glu Gly Asn Val Phe Ser Cys Ser Val Met His Glu
Ala Leu His Asn His Tyr Thr Gln Lys Ser Leu Ser Leu Ser Leu Gly
LC
(SEQ ID NO: 18)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Glu Ser Trp Pro Leu His Phe Gly Gly Gly Thr Lys Val Glu Ile Lys
Arg Thr Val Ala Ala Pro Ser Val Phe Ile Phe Pro Pro Ser Asp Glu Gln Leu Lys Ser Gly Thr
Ala Ser Val Val Cys Leu Leu Asn Asn Phe Tyr Pro Arg Glu Ala Lys Val Gln Trp Lys Val
Asp Asn Ala Leu Gln Ser Gly Asn Ser Gln Glu Ser Val Thr Glu Gln Asp Ser Lys Asp Ser
Thr Tyr Ser Leu Ser Ser Thr Leu Thr Leu Ser Lys Ala Asp Tyr Glu Lys His Lys Val Tyr Ala
Cys Glu Val Thr His Gln Gly Leu Ser Ser Pro Val Thr Lys Ser Phe Asn Arg Gly Glu Cys
HC
(SEQ ID NO: 19)
Glu Val Gln Leu Val Glu Ser Gly Gly Gly Leu Val Gln Pro Gly Gly Ser Leu Arg
Leu Ser Cys Ala Ala Ser Gly Phe Thr Phe Ser Ser Tyr Thr Met Ser Trp Val Arg Gln Ala Pro
Gly Lys Gly Leu Glu Trp Val Ala Thr Ile Ser Gly Gly Gly Phe Gly Thr Tyr Tyr Pro Asp Ser
Val Lys Gly Arg Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Pro Gly Tyr His Asn Tyr Tyr
Phe Asp Ile Trp Gly Gln Gly Thr Thr Val Thr Val Ser Ser Ala Ser Thr Lys Gly Pro Ser Val
Phe Pro Leu Ala Pro Cys Ser Arg Ser Thr Ser Glu Ser Thr Ala Ala Leu Gly Cys Leu Val Lys
Asp Tyr Phe Pro Glu Pro Val Thr Val Ser Trp Asn Ser Gly Ala Leu Thr Ser Gly Val His Thr
Phe Pro Ala Val Leu Gln Ser Ser Gly Leu Tyr Ser Leu Ser Ser Val Val Thr Val Pro Ser Ser
Ser Leu Gly Thr Lys Thr Tyr Thr Cys Asn Val Asp His Lys Pro Ser Asn Thr Lys Val Asp
Lys Arg Val Glu Ser Lys Tyr Gly Pro Pro Cys Pro Pro Cys Pro Ala Pro Glu Ala Ala Gly Gly
Pro Ser Val Phe Leu Phe Pro Pro Lys Pro Lys Asp Thr Leu Met Ile Ser Arg Thr Pro Glu Val
Thr Cys Val Val Val Asp Val Ser Gln Glu Asp Pro Glu Val Gln Phe Asn Trp Tyr Val Asp
Gly Val Glu Val His Asn Ala Lys Thr Lys Pro Arg Glu Glu Gln Phe Asn Ser Thr Tyr Arg
Val Val Ser Val Leu Thr Val Leu His Gln Asp Trp Leu Asn Gly Lys Glu Tyr Lys Cys Lys
Val Ser Asn Lys Gly Leu Pro Ser Ser Ile Glu Lys Thr Ile Ser Lys Ala Lys Gly Gln Pro Arg
Glu Pro Gln Val Tyr Thr Leu Pro Pro Ser Gln Glu Glu Met Thr Lys Asn Gln Val Ser Leu Thr
Cys Leu Val Lys Gly Phe Tyr Pro Ser Asp Ile Ala Val Glu Trp Glu Ser Asn Gly Gln Pro Glu
Asn Asn Tyr Lys Thr Thr Pro Pro Val Leu Asp Ser Asp Gly Ser Phe Phe Leu Tyr Ser Arg
Leu Thr Val Asp Lys Ser Arg Trp Gln Glu Gly Asn Val Phe Ser Cys Ser Val Met His Glu
Ala Leu His Asn His Tyr Thr Gln Lys Ser Leu Ser Leu Ser Leu Gly
LC
(SEQ ID NO: 20)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Ala Ser Trp Pro Leu His Phe Gly Gly Gly Thr Lys Val Glu Ile Lys
Arg Thr Val Ala Ala Pro Ser Val Phe Ile Phe Pro Pro Ser Asp Glu Gln Leu Lys Ser Gly Thr
Ala Ser Val Val Cys Leu Leu Asn Asn Phe Tyr Pro Arg Glu Ala Lys Val Gln Trp Lys Val
Asp Asn Ala Leu Gln Ser Gly Asn Ser Gln Glu Ser Val Thr Glu Gln Asp Ser Lys Asp Ser
Thr Tyr Ser Leu Ser Ser Thr Leu Thr Leu Ser Lys Ala Asp Tyr Glu Lys His Lys Val Tyr Ala
Cys Glu Val Thr His Gln Gly Leu Ser Ser Pro Val Thr Lys Ser Phe Asn Arg Gly Glu Cys
LC
(SEQ ID NO: 21)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Glu Gln Gly Ile Pro Ala Arg Phe Ser
Gly Ser Gly Ser Gly Thr Asp Phe Thr Leu Thr Ile Ser Ser Leu Glu Pro Glu Asp Phe Ala Val
Tyr Tyr Cys Gln Gln Ser Ala Ser Trp Pro Leu His Phe Gly Gly Gly Thr Lys Val Glu Ile Lys
Arg Thr Val Ala Ala Pro Ser Val Phe Ile Phe Pro Pro Ser Asp Glu Gln Leu Lys Ser Gly Thr
Ala Ser Val Val Cys Leu Leu Asn Asn Phe Tyr Pro Arg Glu Ala Lys Val Gln Trp Lys Val
Asp Asn Ala Leu Gln Ser Gly Asn Ser Gln Glu Ser Val Thr Glu Gln Asp Ser Lys Asp Ser
Thr Tyr Ser Leu Ser Ser Thr Leu Thr Leu Ser Lys Ala Asp Tyr Glu Lys His Lys Val Tyr Ala
Cys Glu Val Thr His Gln Gly Leu Ser Ser Pro Val Thr Lys Ser Phe Asn Arg Gly Glu Cys
Human DKK1 Amino Acid Sequence
(SEQ ID NO: 22)
Thr Leu Asn Ser Val Leu Asn Ser Asn Ala Ile Lys Asn Leu Pro Pro Pro Leu Gly
Gly Ala Ala Gly His Pro Gly Ser Ala Val Ser Ala Ala Pro Gly Ile Leu Tyr Pro Gly Gly Asn
Lys Tyr Gln Thr Ile Asp Asn Tyr Gln Pro Tyr Pro Cys Ala Glu Asp Glu Glu Cys Gly Thr
Asp Glu Tyr Cys Ala Ser Pro Thr Arg Gly Gly Asp Ala Gly Val Gln Ile Cys Leu Ala Cys
Arg Lys Arg Arg Lys Arg Cys Met Arg His Ala Met Cys Cys Pro Gly Asn Tyr Cys Lys Asn
Gly Ile Cys Val Ser Ser Asp Gln Asn His Phe Arg Gly Glu Ile Glu Glu Thr Ile Thr Glu Ser
Phe Gly Asn Asp His Ser Thr Leu Asp Gly Tyr Ser Arg Arg Thr Thr Leu Ser Ser Lys Met
Tyr His Thr Lys Gly Gln Glu Gly Ser Val Cys Leu Arg Ser Ser Asp Cys Ala Ser Gly Leu
Cys Cys Ala Arg His Phe Trp Ser Lys Ile Cys Lys Pro Val Leu Lys Glu Gly Gln Val Cys Thr
Lys His Arg Arg Lys Gly Ser His Gly Leu Glu Ile Phe Gln Arg Cys Tyr Cys Gly Glu Gly
Leu Ser Cys Arg Ile Gln Lys Asp His His Gln Ala Ser Asn Ser Ser Arg Leu His Thr Cys Gln
Arg His

### Exemplification

Description of Clinical Trials

The data discussed in detail below resulted from three separate clinical trials. The three separate clinical trials were differentiated by the type of cancer being treated. The three trials were directed to treatment of esophagogastric cancer, gynecological cancer and biliary tract cancer. In each study, patients were administered 300 mg of DKN-01, with the exception of two patients that received 150 mg of DKN-01, either alone or in combination with at least one other additional therapeutic agent. Each study is discussed in detail below.

Esophagogastric Cancer

Subjects: Male and female patients having histologically confirmed recurrent or metastatic esophageal or gastroesophageal junction (GEJ) or gastric adenocarcinoma.

Treatment Regimens:
a) Combination Therapy-DKN-01 and paclitaxel, 28-day cycle: 300 mg of DKN-01 was administered IV over a minimum of 30 minutes and up to a maximum of 2 hours given on days 1 and 15 of each cycle without interruption plus paclitaxel administered IV over 1 hour on Days 1, 8, 15 and 22 according to standard clinical practice. DKN-01 was administered first followed by paclitaxel. Dose of DKN-01 was 300 mg. Dose of paclitaxel was 80 mg/m².
b) Combination Therapy-DKN-01 and pembrolizumab, 21-day cycle: 300 mg of DKN-01 was administered IV over a minimum of 30 minutes and up to a maximum of 2 hours given on Days 1 and 15 of each cycle without interruption plus pembrolizumab administered IV over 30 minutes on Days 1 of each cycle according to standard clinical practice. Two patients received 150 mg DKN-01. DKN-01 was administered first followed by pembrolizumab.
c) Monotherapy-DKN-01, 28-day cycle: 300 mg of DKN-01 was administered IV over a minimum of 30 minutes and up to a maximum of 2 hours given on Days 1 and 15 of each cycle.

The patient's duration of study participation includes a Screening Period, a Treatment Period (either a 21-day cycle or 28-day cycle as noted above) and a Follow-up Period. For the Follow-up Period, a visit was scheduled within 30 days after the last dose of study treatment. Patients had a physical examination, concomitant medication review, ECOG PS, vital signs, clinical safety laboratory tests, serum pregnancy test, 12-lead ECG, solid tumor measurement and radiologic assessment are performed at this visti. A single blood sample for pharmacokinetic and pharmacodynamics evaluation was taken as a random sample. Long-term follow-up after study treatment discontinuation, patients who do not have progressive disease continue to be monitored every 12 weeks per routine clinical practice including evaluation for tumor response until disease progressing using RECIST v1.1 guidelines, death, or until study closure.

### Efficacy Evaluation:

The antitumor activity of the combination therapy was accessed as follows.

Each patient was assessed by one or more of the following radiologic tests for tumor measurement:
Computed tomography (CT) scan
MRI
Chest x-ray
CT/positron emission tomography (CT/PET) scans (whenever feasible, the preferred imaging modality to be used throughout the study is CT/PET scans).

A baseline radiological assessment of the solid tumor(s) was done and repeated prior to the start of Cycle 3 and every odd cycle thereafter to evaluate for tumor response and progression using RECIST v. 1.1 (Revised RECIST Guideline Version 1.1; Eisenhauer et al. 2009). For patients receiving combination therapy of DKN-01 and pembrolizumab, tumor response and progression was assessed in some instances uding the Immune-related Response Criteria (iRECIST) (See, Seymour L, Bogaerts J, Perrone A, et al. iRECIST: Guidelines for response criteria for use in trials testing immunotherapeutics. Lancet Oncol 2017; 18(3):e143-e152).

Evaluation of target lesions were characterized as follows according to RECIST v. 1.1:

Complete Response (CR): Disappearance of all target lesions. Any pathological lymph nodes (whether target or non-target) must have reduction ins short axis to <10 mm. tumor marker results must have normalized.

Partial Response (PR): At least a 30% decrease in the sum diameter of target lesions, taking as reference the baseline sum diameters.

Progressive Disease (PD): At least a 20% increase in the sum of the diameters of target lesions, taking as reference the smallestsum on study (including the baseline sum if that is the smallest). In addition to the relative increase of 20%, the sum must also demonstrate an absolute increase of at least 5 mm. The appearance of one or more new lesions is also considered progression.

Stable Disease (SD): Neither sufficient shrinkage to qualify for PR nor sufficient increase to qualify for PD, taking as reference the smallest sum diameters while on study.

Not Evaluable (NE): When an incomplete radiologic assessment of target lesions is performed or there is a change in the method of measurement from baseline. (NE may also refer to Not Done/Missing.)

Gynecological Cancers

Subjects: Female patients having Epithelial Endometrial Cancer (EEC) and Epithelial Ovarian Cancer (EOC). Patients with EEC must have a histologically confirmed diagnosis (by either primary surgical specimen or biopsy for recurrence) of recurrent previously treated EEC. Patients with EOC must have a histologically confirmed diagnosis (by either primary surgical specimen or biopsy for recurrence) of recurrent platinum-resistant/refractory EOC, primary peritoneal, or fallopian tube cancer (i.e., disease recurrence within 6 months of completion of or progression during platinum-based chemotherapy).

Treatment Regimens:
a) DKN-01 Monotherapy-28 day cycle: 300 mg DKN-01 on day 1 and day 15 of the 28-day cycle. DKN-01 was administered intravenously over a minimum of 30 minutes and up to a maximum of 2 hours.
b) Combination Therapy-DKN-01 and paclitaxel, 28-day cycle: 300 mg of DKN-01 and 80 mg/m². 300 mg of DKN-01 was administered intravenously over a minimum of 30 minutes and up to a maximum of 2 hours given on day 1 and day 15 of the 28-day cycle. Paclitaxel was administered intravenously over 1 hour on days 1, 8 and 15 of each 28-day cycle according to standard clinical practice. DKN-01 was administered first followed by paclitaxel as separate infusions on day 1 and day 15 of each cycle.

The patient's duration of study participation includes a Screening Period, a Treatment Period and a Follow-up Period. For the Follow-up Period, a visit was scheduled within 30 days after the last treatment administration in the treatment period. After discontinuation of treatment and radiographic documentation of Progressive Disease, all patients will be followed in the survival follow-up phase for survival until death, withdrawal of consent, loss to follow-up, or closure of the study. Survival follow-up will occur 4 times per year (every 3 months) after the end of treatment visit.

Efficacy Evaluation:

The primary efficacy endpoint for each study was Objective Response Rate (ORR) as assessed by using the Response Evaluation Criteria in Solid Tumors, version 1.1 (RECIST 1.1) (Eisenhauer E A, Therasse P, Bogaerts J. et al. New response evaluation criteria in solid tumours: revised RECIST guideline (version 1.1). Eur J Cancer. 2009; 45(2):228-247. ORR is the best overall response [BOR] of complete response [CR]+ partial response [PR]).

Secondary efficacy endpoints in each study were:
a) Objective Disease Control Rate (ODCR) as assessed using RECIST 1.1. ODCR is the CR+PR+stable [SD]>6 weeks (CR=Complete Response and PR=Partial Response);
b) OS (Overall Survival), defined as the time from first study drug dose to death from any cause;
c) PFS (Progression Free Survival), defined as the time from first study drug dose to first radiographically-documented Progressive Disease (PD) as determined using RECIST 1.1 or death due to any cause.
d) TTP, defined as the time from first study drug dose until the date of first radiographically-documented Progressive Disease as determined using RECIST 1.1;
e) DoR (duration of response), defined as the time from initial response (≥PR) until radiographically-documented PD or death; PD is defined using RECIST 1.1;
f) DoCR (duration of complete response), defined as the time from initial CR until radiographically-documented PD or death; PD is defined using RECIST 1.1;
g) DoCB (duration of clinical benefit), defined as the time from the first tumor assessment of CR, PR or SD to the time of PD, as determined using RECIST 1.1, to death due to any cause; and
h) TTTF, defined as the time from first study drug dose until the date of discontinuation of DKN-01 for any reason, including PD, toxicity, and death.

Biliary Tract Cancers

Subjects: Male and female patients having histologically or cytologicallly documented carcinoma primary to the intra- or extra-hepatic biliary system or gall bladder with clinical and/or radiologic evidence of unresectable, locally advanced or metastatic disease.

Treatment Regimen: Combination therapy-DKN-01, gemcitabine and cisplatin, 21-day cycle: 300 mg of DKN-01 was administered IV over a minimum of 30 minutes and up to a maximum of 2 hours given on days 1 and 8 of the 21-day cycle without interruption. On days 1 and 8, cisplatin and gemcitabine was administered via IV infusion according to standard clinical practice. 25 mg/m² of cisplatin is administered and 1000 mg/m² of gemcitabine is administered.

The patient's duration on studying participation includes a Screening Period, a Treatment Period consisting of 21-day cycles and a Post-Treatment Period. Subsequent to completion of the Treatment Period patients are followed via clinic visit or telephone per clinical practice (if the patient discontinued study drug for a reason unrelated to progressive disease) and for survival until death or study closure. Patients who discontinue study treatment without documented disease progression continue to be evaluated for response using RECIST criteria until disease progression, death, or until study closure.

Efficacy Evaluation:

Standard of care radiographic imaging to allow for determination of measureable disease in patients with advanced solid malignancies (e.g., computed tomography [CT], magnetic resonance imaging [MRI]). A baseline radiological assessment of the solid tumor was done and repeated prior to the start of Cycle 3 and every odd numbered cycle thereafter (within 3 days of starting the cycle), to evaluate tumor response using RECIST criteria. Patients should have the same radiographic imaging modality used throughout the study (at baseline and at subsequent assessments) in order to provide uniformity to radiographic assessments. If a response is noted, a follow-up radiographic assessment at a minimum of 4 weeks later to confirm response is required.

Clinical response to treatment was based upon the RECIST 1.1 criteria for solid tumors. The following evaluations will be performed: time to first and best response, objective response rate (ORR), progression free survival (PFS), duration of response (DoR, and overall survival (OS).

### EXAMPLES

### Example 1: RNAscope®-Based Determination of H-Scores and % Positive Values

The H-scores employed in the analysis described herein were obtained as follows.

Single-plex automated RNAscope assays were performed using the 2.5 LS or 2.5 LSx Red Reagent Kit (ACD) on the Leica Biosystems BOND RX platform (LS). This was followed by pretreatment including target retrieval (15 min at 95° C. for using Leica Epitope Retrieval Buffer 2) and protease III treatment for 15 min at 40° C. Probes were then hybridized for 2 h at 42° C. followed by RNAscope specific amplification. The chromogenic detection was then performed using Bond Polymer Refine Red Detection kit. RNAscope probe design has been described previously. The following ACD RNAscope probes were used in this study: Hs-DKK1 (cat. 421418), Hs-PPIB (cat. 313908) and dapB (cat. 312038). The slides were counterstained on the Leica instrument using hematoxylin and the bluing of the counterstain was done offline. The staining was observed as punctate red dots. The slides were imaged using the AT2 scanner from Leica with a 40× objective.

Each sample was quality controlled for RNA integrity with a probe specific to the moderately expressed housekeeping gene PPIB and for background with a probe specific to bacterial dapB RNA. For control probes a semi-quantitative scoring criteria was applied to assess RNA integrity and background staining. Visual (manual) scoring was performed by a qualified scientist at ACD. A score of 0-4 was assigned for each control probe based on the following:
Score 0=No staining or <1 dot/10 cells
Score 1=1-3 dots/cell
Score 2=4-9 dots/cell, no or very few dot clusters
Score 3=10-15 dots/cell and/or <10% dots are in clusters
Score 4=>15 dots/cell and/or >10% dots are in clusters

Samples were considered a quality control (QC) pass if the following criteria were met: 1) The PPIB score was 2 with relatively uniform positive control signal throughout the sample indicating RNA integrity. 2) The dapB score was <1 indicating minimal background signal.

Scanned slide images from DKK1 stained biopsy slides were reviewed by a veterinary pathologist and analyzed using QuPath open-source morphometric analysis program (Bankhead, P. et al. QuPath: Open source software for digital pathology image analysis. Sci Rep 7, 16878, doi:10.1038/s41598-017-17204-5 (2017), incorporated herein by reference). The region(s) of the tissue containing the neoplasm was identified as the region of interest (ROI). The level of DKK1 mRNA expression was calculated as follows.

DKK1 mRNA expression for each neoplastic cell was assigned to the following bins
Bin-0=0 dots/cell
Bin-1=1-3 dots/cell
Bin-2=4-9 dots/cell
Bin-3=10+ dots/cell

For H-score determination, after all neoplastic cells were assigned a score, the percentage scoring in Bin-0, Bin-1, Bin-2, or Bin-3 were used to calculate the H-score:

H-score=(% Bin-3×3)+(% Bin-2×2)+(% Bin-1×1)+(% Bin-0×0)

By this calculation, H-score can range from 0 to 300.

For % Positive determination, after all neoplastic cells were assigned a score, the percentage of positive tumor cells were determined by adding up the total neoplastic cells with staining. This could be done either by adding (Bin-1+Bin-2+Bin-3) and dividing by the total number of neoplastic cells (Bin-0+Bin-1+Bin-2+Bin-3) or by counting all cell assigned as “positive” (e.g., by detecting at least one single staining dot) and dividing this number by the total number of neoplastic cells. % Positive can range from 0 to 100.

For H-score determination, if the QuPath software was unable to identify tumor cells then the slide was manually analyzed by estimating the percentage of cells in Bin-1, Bin-2 and Bin-3, using the same H-score formula as described above.

For % Positive determination, if the QuPath software was unable to identify tumor cells then the slide was manually analyzed by estimating the percentage of cells in Bin-1, Bin-2 and Bin-3. % Positive was calculated by adding the estimated percentage of cells in Bin-1, Bin-2 and Bin-3.

### Example 2: DKK1 Expression H-Score and Clinical Outcome Correlates

1. Patients Having the H-Score in the Top Tertile had Longer Progression Free Survival

In this study, RNAscope data collected from a total of 134 patients that were treated with DKN-01 at 300 mg across various indications and additional combinations. The data was obtained based on the clinical studies described above. Briefly, study P102 involved 67 patients suffering from Esophagogastric cancers (who had tumoral DKK1 mRNA expression assessed), of which 57 patients were treated by a combination of are DKN-01/Keytruda. Of these 57 patients, 31 patients were diagnosed with a gastroesophageal junction cancer and were immunotherapy-naïve (i.e. had not been previously treated by Keytruda or other anti-PD-1/anti-PD-L1 antibodies). Study P204 involved 54 patients suffering from gynecological cancers (who had tumoral DKK1 mRNA expression assessed): 32 patients diagnosed with endometrial cancer (EEC/Epithelial Endometrial Cancer) and 22 patients diagnosed with ovarian cancer (EOC/Epithelial Ovarian Cancer). Study P103 involved 13 patients suffering from biliary tract cancer (cholangiocarcinoma).

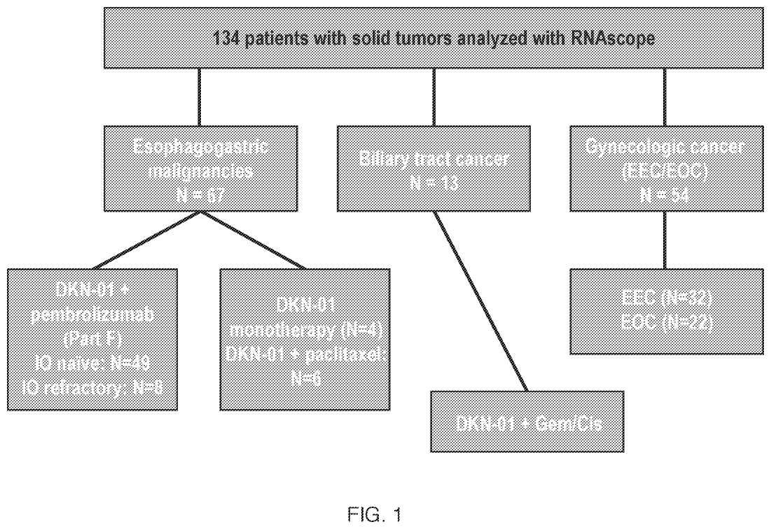

The subgroups of the 134 patients analyzed in this study are graphically represented in FIG. 1

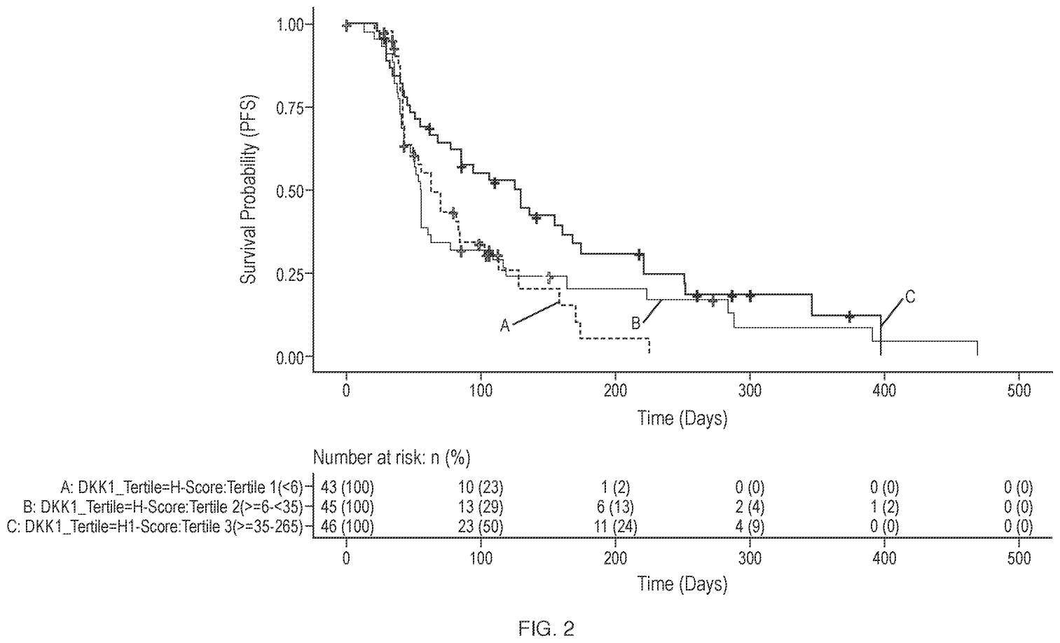

Statistical analysis of the entire 134 patient pool demonstrated that patients having the H-score in the top tertile had longer Progression Free Survival compared to patients having the DKK1 H-score in the lower-tertile or middle-tertile groups. These results are graphically represented in FIG. 2.

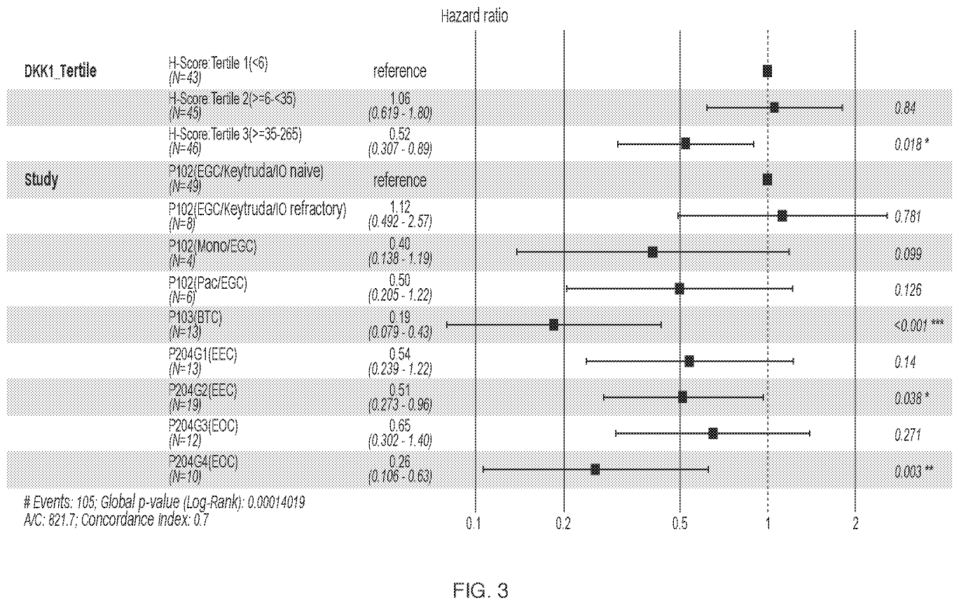

The Hazard Ratio (HR, the risk of having an event that is either “radigographic progression” or “dying” from any cause) was also computed for each tertile as well as for subgroups of patients adjusted for the cancer type and the therapeutic agent(s). The results are graphically represented in FIG. 3. As can be seen, patients having their DKK1 H-score in the upper-tertile had statistically significantly longer PFS compared to lower tertiles, both in general and when adjusted for cancer type and therapeutic agent(s). (The rightmost column of numbers represents P-values of comparison of the HR of each subgroup to the reference subgroup: the bottom tertile of patients by their DKK1 H-score.)

2. Patients Having the H-Score Above an Optimal Cutoff had Longer Progression Free Survival

It was further demonstrated that an optimal cutpoint (referred to herein as an “optimal DKK1 expression H-score”) exists with respect the DKK1 H-score of a cancer patient group, such that when the patients in the subgroup having an H-score above the “optimal cutpoint” are administered a DKK1 inhibitor (e.g. DKN-01), these patients show a statistically significant improvement of the progression-free survival (PFS) as compared to the subgroup of patients having their H-scores at or below the “optimal cutpoint” value.

In the first instance, the pool of 134 cancer patients described above was analyzed using the Standardized Log-Rank Statistics, as described hereinabove.

It was discovered that a value of DKK1 H-score exists that corresponds to the most significant relationship with the survival outcome (PFS). The value of the DKK1 H-score of 38 was obtained for the pooled group. This value of the DKK1 H-score corresponds to the maximum of the standardized log-rank statistic, and is the cutpoint between two subgroups of the entire pool that is most significantly associated with longer PFS.

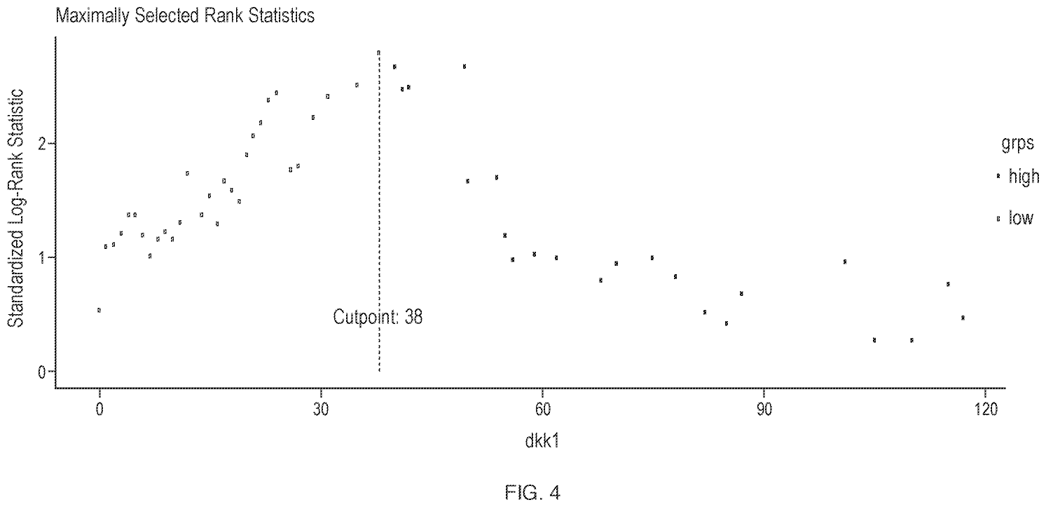

The existence of this maximum of the standardized log-rank statistic is graphically illustrated in FIG. 4.

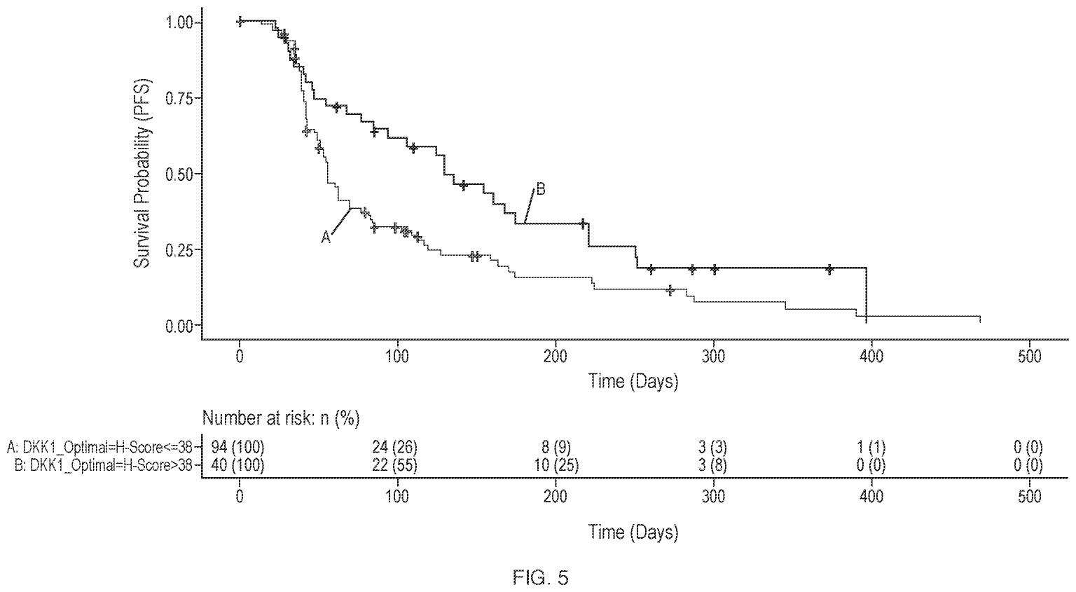

The favorable clinical outcome (longer PFS) of DKN-01 treatment of patients having the DKK1 H-score above the “optimal score” is further illustrated in FIG. 5. FIG. 5 is a superposition of two plots. Each plot represents PFS (expressed as probability) as a function of time. One plot represents the subgroups of patients having the DKK1 H-score above the “optimal” score of 38, and the other—at or below the “optimal” score.

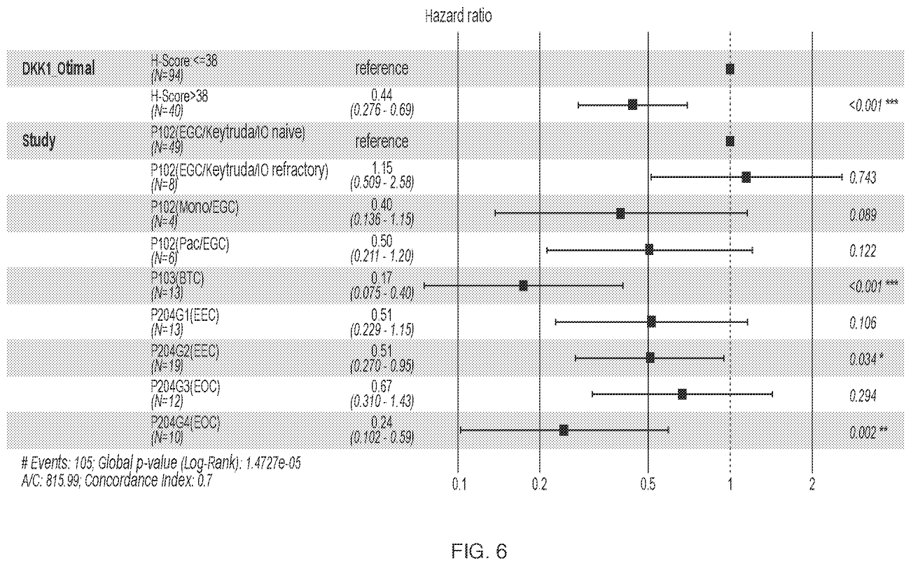

The Hazard Ratio (HR, the risk of having an event that is either “radigographic progression” or “dying” from any cause) was also computed for the group of patients having their H-score above the “optimal” score as compared to the subgroup of patients having their H-scores at or below the “optimal” score. HR for subgroups of patients adjusted for the cancer type and the therapeutic agent(s) were also computed. The results are graphically represented in FIG. 6. As can be seen, patients having their DKK1 H-score above the “optimal” score had statistically significantly lower HR compared to patients at or below the optimal score, when adjusted for cancer type and therapeutic agent(s). (The rightmost column of numbers represents P-values of comparison of the HR of each subgroup to the reference subgroup: the subgroup of patients having their DKK1 H-score at or below the “optimal” score.)

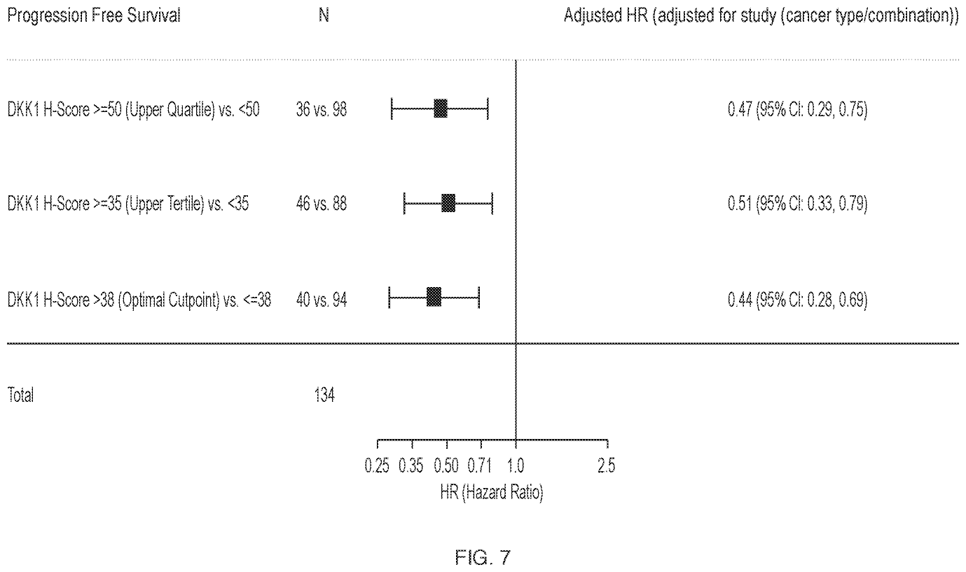

A comparison of different cutpoints demonstrates that optimal cutpoint has the best adjusted Hazard Ration (HR). The results are graphically presented in FIG. 7. As can be seen, the subgroup defined by the H-scores above the “optimal cutpoint” had the best adjusted HR relative to the subgroups defined by either upper tertile or upper quartile. The right column represents HR values and provides 95% confidence intervals.

3. The Value of the Optimal Cutpoint is Similar for Different Cancers

It was discovered that the similar values of the “optimal cutpoint” are derived when the statistical analysis is conducted on patient subgroups defined by different cancers, even after adjustment for the regimen and cancer type.

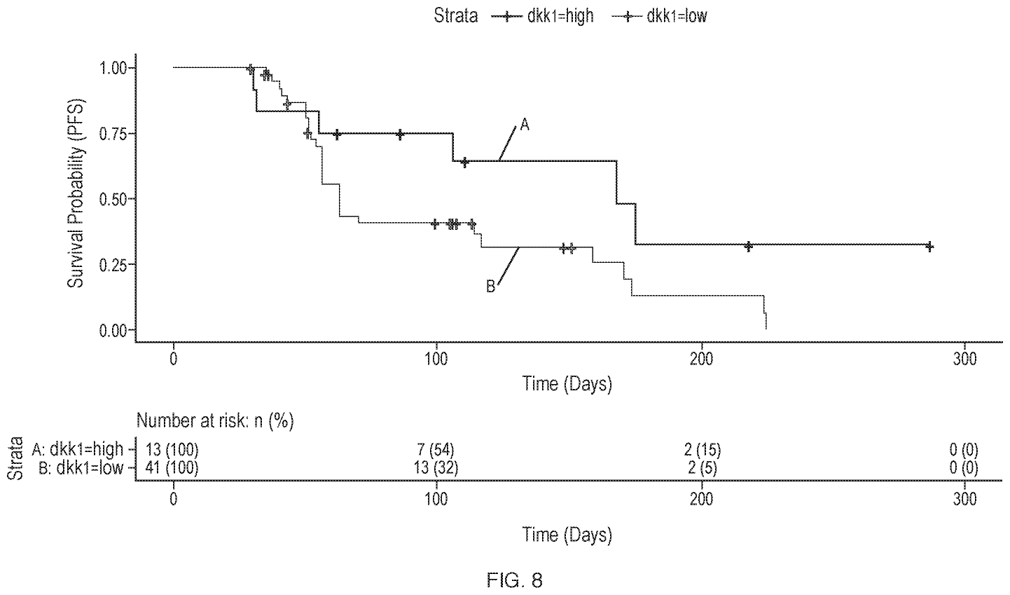

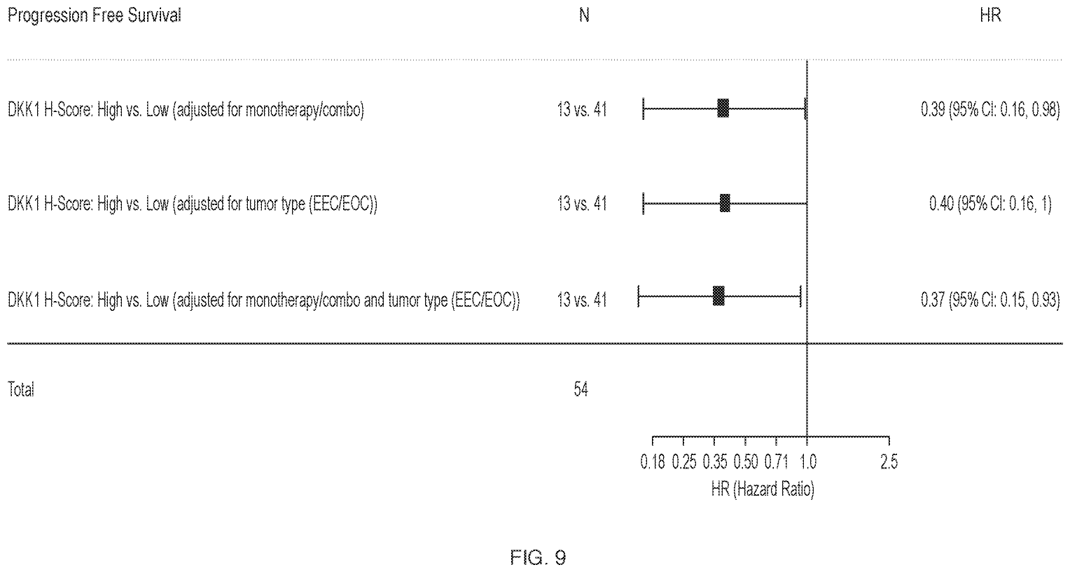

Specifically, it was discovered that DKK1 H-score of above 38 is associated with longer PFS in EEC/EOC. The results are graphically presented in FIG. 8 and FIG. 9. FIG. 8 shows two superimposed plots, each representing PFS (expressed as probability) of a subgroup of EEC/EOC patients. One subgroups has the DKK1 H-scores above the optimal cutpoint, the other subgroup is at or below this optimal cutpoint. FIG. 9 shows a table and a plot representing HR for the sub-subgroups of EEC/EOC patients. As can be seen, the HR values for the three adjusted subgroups are similar. The right column represents HR values and provides 95% confidence intervals.

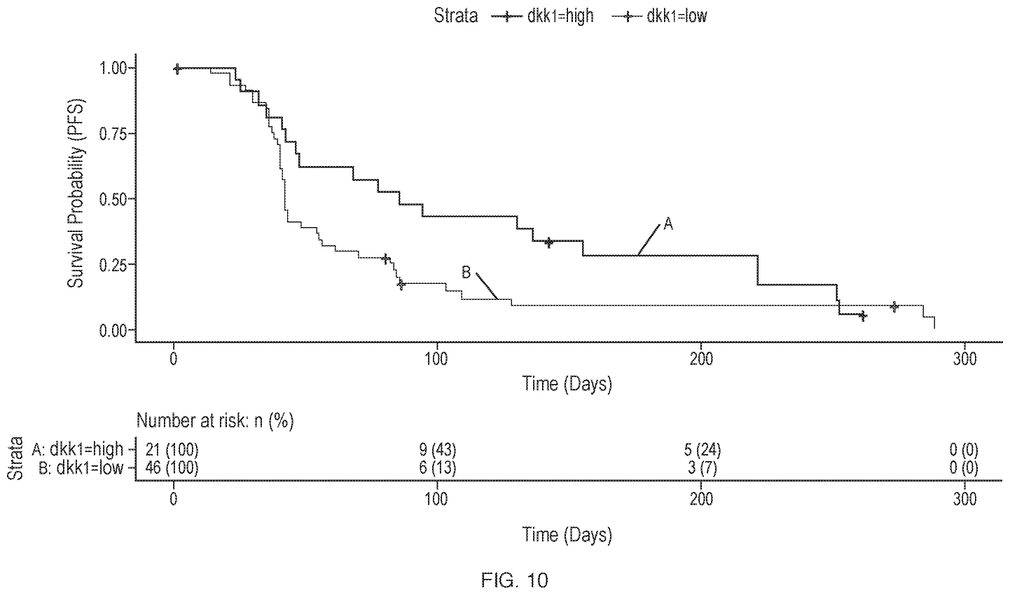

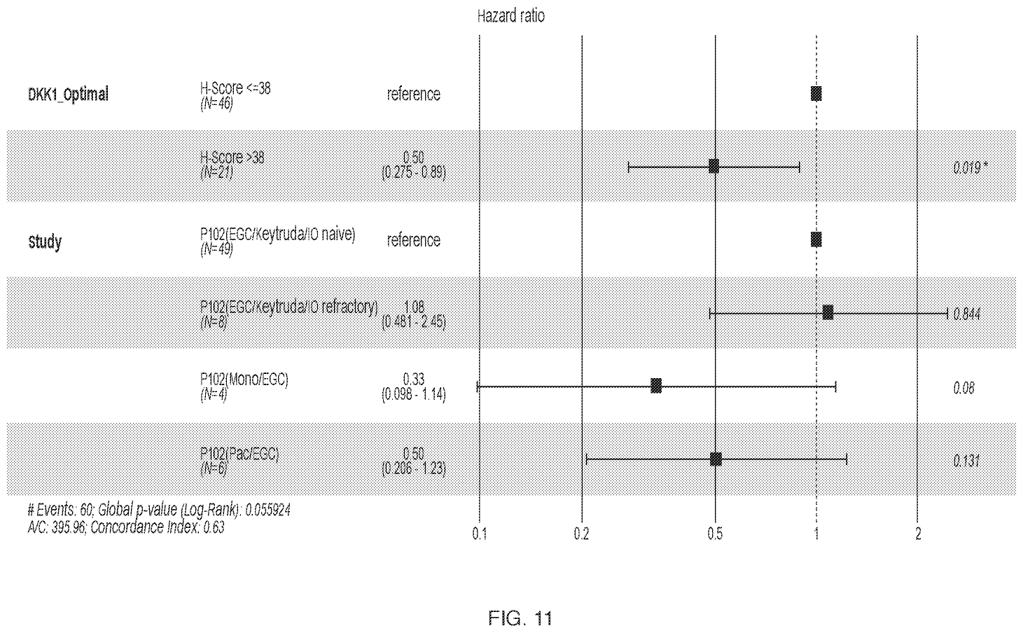

In a further study, it was discovered that DKK1 H-score of above the “optimal cutpoint” value of 38 is associated with longer PFS in GEJ/GC/EC patients. The data was pooled for 67 esophagogastric cancer patients receiving either DKN-01 monotherapy, or a combination therapy of DKN-01 and either Keytruda or paclitaxel. The results are graphically presented in FIG. 10 and FIG. 11.

FIG. 10 shows two superimposed plots, each representing PFS (expressed as probability) of a subgroup of GEJ/GC/EC patients. One subgroup has the DKK1 H-scores above the optimal cutpoint of 38, the other subgroup at or below this optimal cutpoint. FIG. 11 shows a table and a plot representing HR for the same sub-subgroups of GEJ/GC/EC patients as discussed with reference to FIG. 10. As can be seen, the subgroup defined by the H-scores above the “optimal cutpoint” had a statistically significantly lower HR compared to patients at or below the optimal cutpoint when adjusted for cancer type and therapeutic agent. The right column represents HR values and provides 95% confidence intervals.

4. GEJ/Gastric Cancer Patients Having an H-Score in the Top Tertile had Longer Progression Free Survival when Treated with DKN-01 and an Anti-PD-1 Antibody

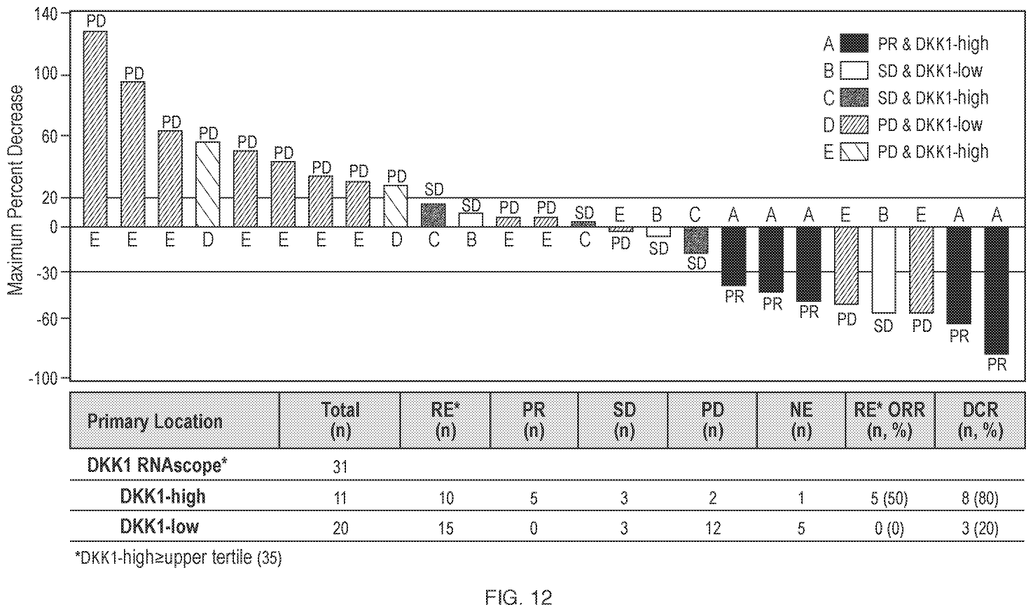

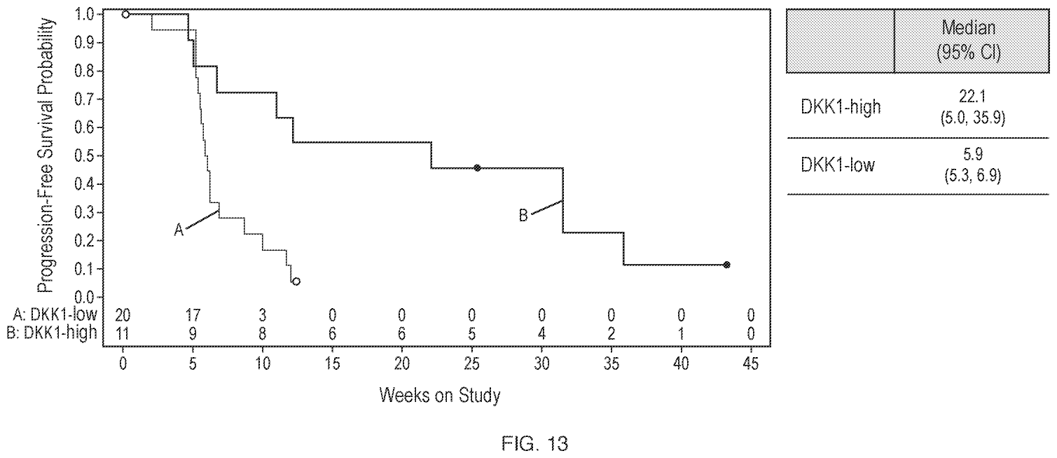

In a further study, out of the patients described above with respect to FIGS. 10 and 11, a subgroup of 31 patients (25 of which were evaluable) was selected. These patients suffered from higher gastro-esophageal junction cancer or gastric cancer and had never been treated with an immunotherapy (so-called, “IO-naïve patients”). As the bar plot presented in FIG. 12 indicates, patients that demonstrated Partial response had the DKK1 H-score in the upper tertile (here, H-score of 35 and above), whereas most patients that exhibited progressive disease had a DKK1 H-score below the upper tertile. A progression-free survival probability (PFS) analysis of the subgroups of 31 patients indicated that median PFS of patients in the upper tertile of DKK1 H-score (H-score of 35 or above) was statistically meaningfully longer at 22.2 weeks compared to the patients having DKK1 H-score below the upper tertile (5.9 weeks). The results of this analysis are presented in FIG. 13.

### Example 3: DKK1 Staining % Positive Values and Clinical Outcome Correlate

In a further study, the RNAscope data of 69 esophagogastric cancer (EGC) patients receiving DKN-01 monotherapy or a combination of DKN-01 at 300 mg and a second agent, as described with respect to FIG. 1 (plus 2 additional patients receiving DKN-01 at 150 mg and pembrolizumab) was analyzed.

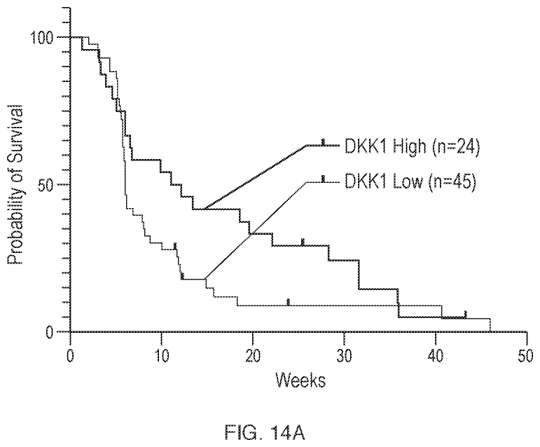

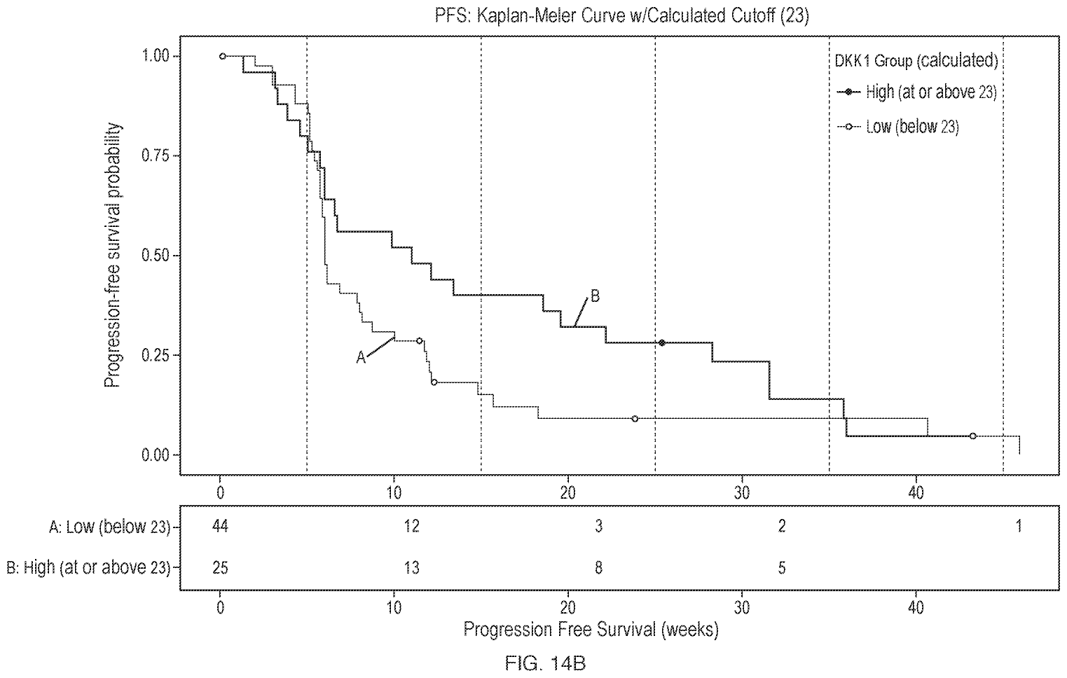

The PFS of the patients was determined using % positive measure. It was discovered that EGC patients having a higher percentage of tumor cells staining positive for DKK1 exhibit an increased PFS. Specifically, the upper tertile of patients (“DKK1 High,” those where at least 26% of tumor cells stained positive for DKK1 by RNAscope) had a median survival of 11.6 weeks, whereas the patients below the upper tertile had a median survival of 6.0 weeks, with the p-value=0.08 by Log rank (Mantel-Cox) test. The results of this analysis (cutoff value of 26%, corresponding to the upper tertial) are graphically presented in FIG. 14A.

The “optimal” cutoff value for the same 69 patients was also calculated and the PFS plot generated.

Briefly, the log-rank statistic for all potential cut points (at most equal to the number of samples in the analysis) was first calculated. The method described in Hothorn and Lausen (Science Direct Working Paper No S1574-0358(04)70152-5, 5 Mar. 2018, available at https://papers.ssm.com/sol3/papers.cfm?abstract_id=3133711) was applied in order to select the cut point. This was done by computing the upper bound of the p-value using the exact distribution of the log-rank statistic under the null hypothesis (HO: survival is independent of the membership in the groups defined by the cut point in the continuous variable, here: % positive is positive).

Here, the optimal cutoff value by the % positive measure was 23%. Median survival for DKK1-high patients (% positive at or above 23%) was 11 weeks (95% CI 6.000-28.286), median survival for DKK1-low patients (% positive below 23%) was 6 weeks (95% CI 5.857-8.714). P-val=0.116. The PFS plot is presented in FIG. 14B.

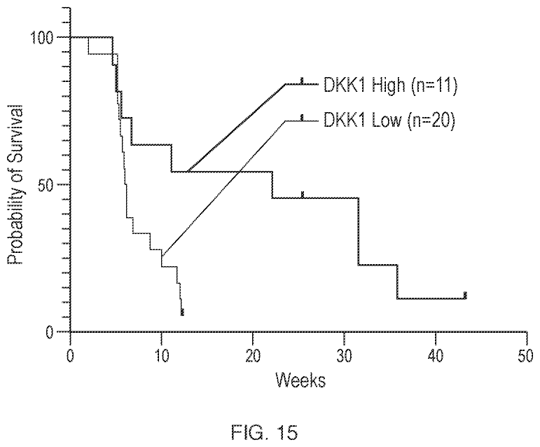

In a further study, the same sub-group of patients as discussed above with respect to FIG. 13 (31 Gastric(G)/gastroesophageal junction (GEJ) patients) was analyzed using % Positive values for DKK1 expression. The results are presented in FIG. 15. It was discovered that G/GEJ IO-naïve patients having a higher percentage of tumor cells staining positive for DKK1 exhibit an increased PFS. Specifically, patients in the upper tertle of the % Positive values (at least 20% of tumor cell stained positive for DKK1 by RNAscope) demonstrated a median survival of 22.1 weeks, compared to 6.1 weeks for the patients outside the upper tertile (p-value=0.017 by Log rank (Mantel-Cox) test).

### Example 4

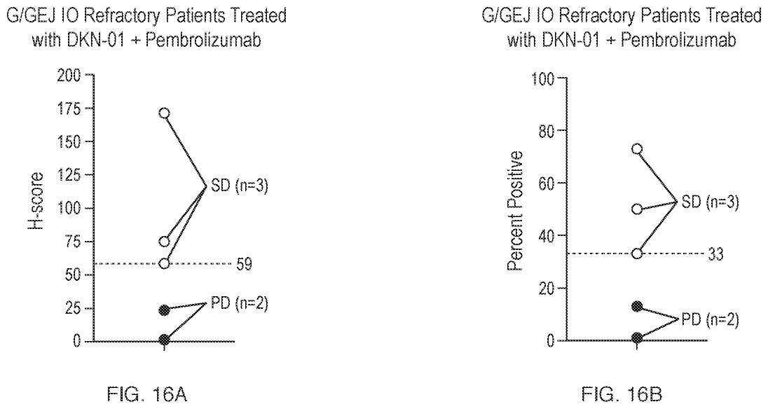

In a further study, a subgroup of patients suffering from Gastric/GEJ IO-Refractory patients (i.e. patients previously treated with immunotherapy) was analyzed. Five patients were treated with DKN-01 in combination with pembrolizumab (4 patients with 300 mg DKN-01 and 1 with 150 mg DKN-01). The results of the analysis are presented in FIG. 16A (for H-score) and FIG. 16B (for % positive values). As can be seen, 3 out of the 5 patients had had stable disease (SD), with 2 patients having progressive disease (PD). All patients with SD had H-scores or % positive values higher than PD patients. The lowest H-score of SD patients was 59 and the lowest % positive value of SD patients was 33%.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a cancer in a subject in need thereof, comprising:
determining a DKK1 expression H-score in a sample of the subject's cancer; and
administering a first amount of a DKK1 inhibitor to the subject determined to have the DKK1 expression H-score above a predetermined value.

2. The method of claim 1, wherein the predetermined value is an optimal DKK1 expression H-score.

3. The method of claim 1, wherein the predetermined value is the lower boundary of the upper tertile of DKK1 expression H-scores in a sample of subjects suffering from cancer.

4. The method of any one of claims 1-3, wherein the cancer is selected from an esophagogastric cancer, a gynecological cancer, or a cholangiocarcinoma.

5. The method of claim 4, wherein the cancer is selected from an esophageal cancer, a gastro-esophageal junction cancer, a gastric cancer, an epithelial endometrial cancer, an epithelial ovarian cancer, or a cholangiocarcinoma.

6. The method of claim 4, wherein the patient is immunotherapy-naïve and is suffering from an esophagogastric cancer.

7. The method of claim 4, wherein the patient is suffering from a gastroesophageal cancer or a gastric cancer.

8. The method of claim 5, wherein the cancer is esophagogastric cancer and is refractory to one or more immunotherapeutic agents.

9. The method of any one of claims 1-4, wherein:
the cancer is the esophagogastric cancer and the predetermined value is the lower boundary of the upper tertile of DKK1 expression H-scores in a sample of subjects suffering from esophagogastric cancer, or
the cancer is the gynecological cancer and the predetermined value is the lower boundary of the upper tertile of DKK1 expression H-scores in a sample of subjects suffering from the gynecological cancer.

10. The method of any one of claims 1-4, wherein:
the cancer is the esophagogastric cancer and the predetermined value is an optimal cutoff value of 38,
the cancer is the gynecological cancer and the predetermined value is an optimal cutoff value of 38, or
the cancer is cholangiocarcinoma, and the predetermined value is an optimal cutoff value of 38.

11. The method of any one of claims 1-4, wherein the cancer is the gastric/GEJ cancer, and the predetermined value is 35.

12. The method of claim 11, wherein the subject is 10-naïve.

13. The method of any one of claims 1-12, wherein the DKK1 inhibitor is a DKK1 antibody or antigen binding-fragment thereof.

14. The method of any one of claims 1-13, wherein the DKK1 antibody, or antigen binding-fragment thereof, comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, LCDR2 has the amino sequence of SEQ ID NO:2, LCDR3 has the amino sequence of SEQ ID NO:3, HCDR1 has the amino sequence of SEQ ID NO:4, HCDR2 has the amino sequence of SEQ ID NO:5, and an HCDR3 has the amino sequence of SEQ ID NO:6.

15. The method of claim 14, wherein the LCVR comprises the amino acid sequence of SEQ ID NO: 7 and the HCVR comprises the amino acid sequence of SEQ ID NO: 8.

16. The method of claim 14 or 15, wherein the LCVR and HCVR comprise amino acid sequences selected from the group consisting of: (i) a LCVR comprising the amino acid sequence of SEQ ID NO: 9 and a HCVR comprising the amino acid sequence of SEQ ID NO: 10; (ii) a LCVR comprising the amino acid sequence of SEQ ID NO: 11 and a HCVR comprising the amino acid sequence of SEQ ID NO: 12; (iii) a LCVR comprising the amino acid sequence of SEQ ID NO: 13 and a HCVR comprising the amino acid sequence of SEQ ID NO: 10; (iv) a LCVR comprising the amino acid sequence of SEQ ID NO: 14 and a HCVR comprising the amino acid sequence of SEQ ID NO: 10.

17. The method of claim 16, wherein the LCVR comprises the amino acid sequence of SEQ ID NO: 11 and the HCVR comprises the amino acid sequence of SEQ ID NO: 12.

18. The method of claim 17, wherein the DKK1 antibody comprises a heavy chain and a light chain amino acid sequence selected from the group consisting of a) a heavy chain comprising the amino acid sequence of SEQ ID NO: 19 and light chain comprising the amino acid sequence of SEQ ID NO: 16, b) a heavy chain comprising the amino acid sequence of SEQ ID NO: 17 and a light chain comprising the amino acid sequence of SEQ ID NO: 18, c) a heavy chain comprising the amino acid sequence of SEQ ID NO: 19 and a light chain comprising the amino acid sequence of SEQ ID NO: 20, and d) a heavy chain comprising the amino acid sequence of SEQ ID NO: 19 and a light chain comprising the amino acid sequence of SEQ ID NO: 21.

19. The method of claim 18, wherein the DKK1 antibody comprises a heavy chain comprising the amino acid sequence of SEQ ID NO: 17 and a light chain comprising the amino acid sequence of SEQ ID NO: 18.

20. The method of any one of claims 1-19, wherein the subject is a human.

21. The method of any one of claims 1-20, further comprising administering to the subject a second amount of a second therapeutic agent.

22. The method of claim 21, wherein the second therapeutic agent is a taxane.

23. The method of claim 22, wherein the second agent is a paclitaxel.

24. The method of claim 21, wherein the cancer is the gynecological cancer and the second therapeutic agent is paclitaxel.

25. The method of claim 24, wherein the cancer is the epithelial endometrial cancer or the epithelial ovarian cancer, the DKK1 antagonist is the DKN-01 antibody, and the second therapeutic agent is paclitaxel.

26. The method of claim 21, further comprising administering to the subject a third amount of a third therapeutic agent.

27. The method of claim 26, wherein the second therapeutic agent is gemcitabine and the third therapeutic agent is a cisplatin.

28. The method of claim 27, wherein the cancer is a biliary tract cancer.

29. A method of treating a cancer in a subject in need thereof, comprising:
determining a DKK1 expression % positive value in a sample of the subject's cancer; and
administering a first amount of a DKK1 inhibitor to the subject determined to have the DKK1 expression % positive value at or above a predetermined value.

30. The method of claim 29, wherein the predetermined value is an optimal value of the DKK1 expression % positive values.

31. The method of claim 29, wherein the predetermined value is the lower boundary of the upper tertile of DKK1 expression % positive values in a sample of subjects suffering from cancer.

32. The method of any one of claims 29-31, wherein the cancer is selected from an esophagogastric cancer, a gynecological cancer, or a cholangiocarcinoma.

33. The method of claim 32, wherein the cancer is selected from an esophageal cancer, a gastro-esophageal junction cancer, a gastric cancer, an epithelial endometrial cancer, an epithelial ovarian cancer, or a cholangiocarcinoma.

34. The method of claim 32, wherein the patient is immunotherapy-naïve and is suffering from an esophagogastric cancer.

35. The method of claim 32, wherein the patient is suffering from a gastroesophageal cancer or a gastric cancer.

36. The method of claim 32, wherein the cancer is esophagogastric cancer and is refractory to one or more immunotherapeutic agents.

37. The method of any one of claims 29-32, wherein:
the cancer is the esophagogastric cancer and the predetermined value is the lower boundary of the upper tertile of DKK1 expression % positive values in a sample of subjects suffering from esophagogastric cancer, or
the cancer is the gynecological cancer and the predetermined value is the lower boundary of the upper tertile of DKK1 expression % positive values in a sample of subjects suffering from the gynecological cancer.

38. The method of any one of claims 29-32, wherein:
the cancer is the esophagogastric cancer and the predetermined value is 26%.

39. The method of any one of claims 29-32, wherein:
the cancer is GC/GEJ and the predetermined value is 20%.

40. The method of claim 39, wherein the subject is IO-naïve.

41. The method of any one of claims 29-40, wherein the DKK1 inhibitor is a DKK1 antibody or antigen binding-fragment thereof.

42. The method of any one of claims 29-41, wherein the DKK1 antibody, or antigen binding-fragment thereof, comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, LCDR2 has the amino sequence of SEQ ID NO:2, LCDR3 has the amino sequence of SEQ ID NO:3, HCDR1 has the amino sequence of SEQ ID NO:4, HCDR2 has the amino sequence of SEQ ID NO:5, and an HCDR3 has the amino sequence of SEQ ID NO:6.

43. The method of claim 42, wherein the LCVR comprises the amino acid sequence of SEQ ID NO: 7 and the HCVR comprises the amino acid sequence of SEQ ID NO: 8.

44. The method of claim 42 or 43, wherein the LCVR and HCVR comprise amino acid sequences selected from the group consisting of: (i) a LCVR comprising the amino acid sequence of SEQ ID NO: 9 and a HCVR comprising the amino acid sequence of SEQ ID NO: 10; (ii) a LCVR comprising the amino acid sequence of SEQ ID NO: 11 and a HCVR comprising the amino acid sequence of SEQ ID NO: 12; (iii) a LCVR comprising the amino acid sequence of SEQ ID NO: 13 and a HCVR comprising the amino acid sequence of SEQ ID NO: 10; (iv) a LCVR comprising the amino acid sequence of SEQ ID NO: 14 and a HCVR comprising the amino acid sequence of SEQ ID NO: 10.

45. The method of claim 44, wherein the LCVR comprises the amino acid sequence of SEQ ID NO: 11 and the HCVR comprises the amino acid sequence of SEQ ID NO: 12.

46. The method of claim 45, wherein the DKK1 antibody comprises a heavy chain and a light chain amino acid sequence selected from the group consisting of a) a heavy chain comprising the amino acid sequence of SEQ ID NO: 19 and light chain comprising the amino acid sequence of SEQ ID NO: 16, b) a heavy chain comprising the amino acid sequence of SEQ ID NO: 17 and a light chain comprising the amino acid sequence of SEQ ID NO: 18, c) a heavy chain comprising the amino acid sequence of SEQ ID NO: 19 and a light chain comprising the amino acid sequence of SEQ ID NO: 20, and d) a heavy chain comprising the amino acid sequence of SEQ ID NO: 19 and a light chain comprising the amino acid sequence of SEQ ID NO: 21.

47. The method of claim 46, wherein the DKK1 antibody comprises a heavy chain comprising the amino acid sequence of SEQ ID NO: 17 and a light chain comprising the amino acid sequence of SEQ ID NO: 18.

48. The method of any one of claims 29-47, wherein the subject is a human.

49. The method of any one of claims 29-48, further comprising administering to the subject a second amount of a second therapeutic agent.

50. The method of claim 49, wherein the second therapeutic agent is a taxane.

51. The method of claim 50, wherein the second agent is a paclitaxel.

52. The method of claim 49, wherein the cancer is the gynecological cancer and the second therapeutic agent is paclitaxel.

53. The method of claim 52, wherein the cancer is the epithelial endometrial cancer or the epithelial ovarian cancer, the DKK1 antagonist is the DKN-01 antibody, and the second therapeutic agent is paclitaxel.

54. The method of claim 50, further comprising administering to the subject a third amount of a third therapeutic agent.

55. The method of claim 54, wherein the second therapeutic agent is gemcitabine and the third therapeutic agent is a cisplatin.

56. The method of claim 55, wherein the cancer is a biliary tract cancer.

57. The method of claim 4, wherein the cancer is gastric/GEJ cancer and is refractory to one or more immunotherapeutic agents.

58. The method of claim 4, wherein the cancer is gastric cancer and is refractory to one or more immunotherapeutic agents.

59. The method of claim 4, wherein the cancer is GEJ cancer and is refractory to one or more immunotherapeutic agents.

60. The method of any one of claims 57-59, wherein the predetermined value is 59.

61. The method of claim 32, wherein the cancer is gastric/GEJ cancer and is refractory to one or more immunotherapeutic agents.

62. The method of claim 32, wherein the cancer is gastric cancer and is refractory to one or more immunotherapeutic agents.

63. The method of claim 32, wherein the cancer is GEJ cancer and is refractory to one or more immunotherapeutic agents.

64. The method of any one of claims 61-63, wherein the predetermined value is 33%.

65. The method of claim 30, wherein the cancer is the esophagogastric cancer (EGC) and the predetermined value is an optimal cutoff value of 23%.

66. A method of treating a cancer in a subject in need thereof, comprising:
determining a DKK1 expression H-score in a sample of the subject's cancer; and
administering a first amount of a DKK1 inhibitor to the subject determined to have the DKK1 expression H-score at or above 30.

67. The method of claim 66, wherein the subject is determined to have the DKK-1 H-score at or above 35.

68. The method of any on of claim 66 or 67, wherein the cancer is GEJ/GC.


SEQUENCE LISTING (22 sequences)

SEQ ID NO: 1 - protein, 11 aa, Artificial Sequence
  source: /note=“Description of Artificial Sequence Synthetic peptide”
HASDSISNSLH

SEQ ID NO: 2 - protein, 7 aa, Artificial Sequence
  source: /note=“Description of Artificial Sequence Synthetic peptide”
  VARIANT (2)..(2): /replace=“Ala”
  VARIANT (6)..(6): /replace=“Glu”
  SITE (1)..(7): /note=“Variant residues given in the sequence have no preference with respect to those in the annotations for variant positions”
YGRQSIQ

SEQ ID NO: 3 - protein, 9 aa, Artificial Sequence
  source: /note=“Description of Artificial Sequence Synthetic peptide”
  VARIANT (4)..(4): /replace=“Ala”
  SITE (1)..(9): /note=“Variant residues given in the sequence have no preference with respect to those in the annotations for variant positions”
QQSESWPLH

SEQ ID NO: 4 - protein, 10 aa, Artificial Sequence
  source: /note=“Description of Artificial Sequence Synthetic peptide”
GFTFSSYTMS

SEQ ID NO: 5 - protein, 16 aa, Artificial Sequence
  source: /note=“Description of Artificial Sequence Synthetic peptide”
TISGGGFGTYYPDSVK

SEQ ID NO: 6 - protein, 10 aa, Artificial Sequence
  source: /note=“Description of Artificial Sequence Synthetic peptide”
  VARIANT (4)..(4): /replace=“Asn”
  SITE (1)..(10): /note=“Variant residues given in the sequence have no preference with respect to those in the annotations for variant positions”
PGYHNYYFDI

SEQ ID NO: 7 - protein, 107 aa, Artificial Sequence
  source: /note=“Description of Artificial Sequence Synthetic polypeptide”
  VARIANT (51)..(51): /replace=“Ala”
  VARIANT (55)..(55): /replace=“Glu”
  VARIANT (92)..(92): /replace=“Ala”
  SITE (1)..(107): /note=“Variant residues given in the sequence have no preference with respect to those in the annotations for variant positions”
EIVLTQSPATLSLSPGERATLSCHASDSISNSLHWYQQKPGQAPRLLIYYGRQSIQGIPARFSGSGSGTDFTLTISSLEPEDFAVYYCQQSESWPLHFGGGTKVEIK

SEQ ID NO: 8 - protein, 119 aa, Artificial Sequence
  source: /note=“Description of Artificial Sequence Synthetic polypeptide”
  VARIANT (102)..(102): /replace=“Asn”
  SITE (1)..(119): /note=“Variant residues given in the sequence have no preference with respect to those in the annotations for variant positions”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYHNYYFDIWGQGTTVTVSS

SEQ ID NO: 9 - protein, 107 aa, Artificial Sequence; identical to SEQ ID NO: 7
  source: /note=“Description of Artificial Sequence Synthetic polypeptide”

SEQ ID NO: 10 - protein, 119 aa, Artificial Sequence; identical to SEQ ID NO: 8
  source: /note=“Description of Artificial Sequence Synthetic polypeptide”

SEQ ID NO: 11 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSIQGIPARFSGSGSGTDFTLTISSLEPEDFAVYYCQQSESWPLHFGGGTKVEIK

SEQ ID NO: 12 - protein, 119 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYNNYYFDIWGQGTTVTVSS

SEQ ID NO: 13 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSASWPLHFGGGTKVEIK

SEQ ID NO: 14 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSEQGIPARFSGSGSGTDFTLTISSLEPEDFAVYYCQQSASWPLHFGGGTKVEIK

SEQ ID NO: 15 - protein, 445 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYHNYYFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSESWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 445 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYNNYYFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSIQGIPARFSGSGSGTDFTLTISSLEPEDFAVYYCQQSESWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 445 aa, Artificial Sequence; identical to SEQ ID NO: 15
  source: /note=“Description of Artificial Sequence Synthetic polypeptide”

SEQ ID NO: 20 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSASWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSEQGIPARFSGSGSGTDFTLTISSLEPEDFAVYYCQQSASWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 235 aa, Artificial Sequence
  source: /note=“Description of Artificial Sequence Synthetic polypeptide”
TLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH